(19) United States
(12) Patent Application Publication
(10) Pub. No.: US 2024/0350493 A1
(43) Pub. Date: Oct. 24, 2024

(54) Methods of Administering Belumosudil in Combination with CYP3A Inducers and/or Proton Pump Inhibitors

(71) Applicant: Kadmon Corporation, LLC, Bridgewater, NJ (US)

(72) Inventors: Olivier Schueller, Cambridge, MA (US); Jeegar P. Patel, Hillsborough, NJ (US)

(73) Assignee: Kadmon Corporation, LLC, Bridgewater, NJ (US)

(21) Appl. No.: 18/644,700

(22) Filed: Apr. 24, 2024

Related U.S. Application Data
(63) Continuation of application No. 18/105,315, filed on Feb. 3, 2023, now abandoned.
Application No. 18/105,315 is a continuation of application No. PCT/US2022/037200, filed on Jul. 14, 2022.

(51) Int. Cl. A61K 31/517; A61K 9/20; A61K 31/4439; A61K 31/496; A61P 37/06

(52) U.S. Cl. CPC A61K 31/517; A61K 9/2009; A61K 9/2013; A61K 9/2027; A61K 9/2031; A61K 9/2054; A61K 31/4439; A61K 31/496; A61P 37/06

(57) ABSTRACT

The present disclosure relates to the co-administration of Belumosudil with CYP3A Inducers and/or Proton Pump Inhibitors for use in the treatment of subjects with chronic graft-versus-host disease (cGVHD).

### CROSS-REFERENCE TO RELATED APPLICATIONS

This application is a continuation of U.S. application Ser. No. 18/105,315, filed Feb. 3, 2023, which is a continuation under 35 U.S.C. § 111 (a) of International Patent Application No. PCT/US2022/037200, filed Jul. 14, 2022, the entire contents of which are incorporated by reference herein for all purposes.

### TECHNICAL FIELD

The present disclosure relates to use of belumosudil or pharmaceutically acceptable salts thereof to treat subjects with chronic graft-versus-host disease (cGVHD), wherein at least one CYP3A Inducer or Proton Pump Inhibitor is co-administered to the subjects.

### BACKGROUND

Chronic graft-versus-host disease (cGVHD) is an immune-mediated inflammatory and fibrotic disorder. It is a potential, serious complication following solid organ transplant and allogeneic hematopoietic cell transplant (alloHCT). cGVHD affects up to 70% of all alloHCT recipients, with an incidence of 20%-50% in children. It is the leading cause of non-relapse mortality beyond 2 years after alloHCT. The estimated prevalence of cGVHD is 14,000 patients in the United States (as of 2016). (Bachier CR et al: Epidemiology and real-world treatment of chronic graft-versus-host disease post allogeneic hematopoietic cell transplantation: A US claims analysis. Presented at ASH 2019, Orlando, FL, Dec. 7-10, 2019) (“Bachier et al.”)

Patients with cGVHD have substantial impairment in quality of life (QOL) as assessed by the Lee Symptom Scale (LSS), which measures the effect of cGVHD on patients' functioning and well-being. It is reported that only one third of patients who have cGVHD and start systemic treatment will be alive, in remission and off immunosuppressive therapy by 5 years. (Lcc SJ et al: Success of immunosuppressive treatments in patients with chronic graft-versus-host disease. Biol Blood Marrow Transpl 24:555-562, 2018) (“Lec et al.”).

The pathophysiology of cGVHD can be separated into three phases: early inflammation because of tissue injury, a dysregulated adaptive immune system, and chronic inflammation and aberrant tissue repair with fibrosis.

First-line therapy for National Institutes of Health (NIH)-defined moderate to severe chronic graft-versus-host disease (cGVHD) is corticosteroids alone or in combination with sirolimus or a calcineurin inhibitor. However, up to 70% of patients require additional lines of therapy. (Bachier CR et al). Furthermore, the long-term use of corticosteroids is associated with significant side effects. (Lee et al).

Management of cGVHD continues to evolve with the advent of targeted therapies. In 2017, the US Food and Drug Administration approved ibrutinib, a Bruton's Tyr kinase inhibitor, for the treatment of adults with cGVHD after failure of one or more 1 systemic lines of therapy. In patients with cGVHD who were required to have either >25% body surface area erythematous rash or an NIH mouth score of >4, a study with ibrutinib reported an overall response rate (ORR) of 67% and a discontinuation rate because of treatment-emergent adverse events (TEAEs) of 43%. (Waller EK, et al: Ibrutinib for chronic graft-versus-host disease after failure of prior therapy: 1-Year update of a phase 1b/2 study. Biol Blood Marrow Transpl 25:2002-2007, 2019).

There remains an opportunity to study other treatment options for patients who have failed ≥1 lines of therapy.

### SUMMARY

The present disclosure relates to 2-{3-[4-(1H-indazol-5-ylamino)-2-quinazolinyl phenoxy}-N- (propan-2-yl) acetamide, or a pharmaceutically acceptable salt thereof (“Compound” or “Belumosudil”), for use in the treatment of chronic graft-versus-host-disease (cGVHD) in a subject, wherein at least one CYP3A Inducer or Proton Pump Inhibitor (PPI) is co-administered to the subject.

The present disclosure also provides a method for treating a subject with cGVHD who is concurrently taking a CYP3A Inducer and/or PPI comprising administering to the subject an adjusted dose of Belumosudil to mediate the exposure reduction effects of the CYP3A Inducers and/or Proton Pump Inhibitors in treating the cGVHD.

The present embodiments can be understood more fully by reference to the detailed description and examples, which are intended to exemplify non-limiting embodiments.

### BRIEF DESCRIPTION OF THE DRAWINGS

FIG. 1 describes the design for the clinical study described in Example 1.

FIG. 2A shows the mean+SD belumosudil pharmacokinetic profiles following administration of 200 mg belumosudil single dose alone (squares), and in combination with itraconazole (circles), rabeprazole (triangles), and rifampicin (diamonds).

FIG. 2B shows the mean+SD belumosudil pharmacokinetic profiles following administration of 200 mg belumosudil BID alone (squares) and in combination with omeprazole (circles). The inset graphs in FIGS. 2A and 2B show log-linear plots with lower error bars excluded.

FIG. 3A shows the mean+SD KD025m2 pharmacokinetic profiles following administration of 200 mg belumosudil single dose alone (squares), and in combination with itraconazole (circles), rabeprazole (triangles), and rifampicin (diamonds).

FIG. 3B shows the mean+SD KD025m2 pharmacokinetic profiles following administration of 200 mg belumosudil BID alone (squares) and in combination with omeprazole (circles). The inset figures for both FIG. 3A and FIG. 3B show log-linear plots with lower error bars excluded.

FIG. 4A shows the mean+SD KD025 ml pharmacokinetic profiles following administration of 200 mg belumosudil single dose alone (squares), and in combination with itraconazole (circles), rabeprazole (triangles), and rifampicin (diamonds).

FIG. 4B shows the mean+SD KD025 ml pharmacokinetic profiles following administration of 200 mg belumosudil BID alone (squares) and with omeprazole (circles). The inset graphs on FIG. 4A and FIG. 4B show log-linear plots with lower error bars excluded.

FIG. 5 shows the design for the clinical study described in Example 2.

FIG. 6 shows the mean+SD belumosudil pharmacokinetic profiles following administration of 200 mg belumosudil under fasted (tablet) and fed (tablet/capsule) conditions. The inset graph in FIG. 6 shows data on a log-linear scale.

### DETAILED DESCRIPTION

### Overview

cGVHD is characterized by an overproduction of proinflammatory cytokines IL-21 and IL-17, as well as overactivation of T follicular helper cells and B cells, which in turn leads to overproduction antibodies. By controlling ROCK2 activity, belumosudil mediates signaling in immune cellular function and fibrotic pathways, thereby alleviating the effects caused by this debilitating disease, such as inflammation of multiple tissues and fibrotic changes that may involve several organs including the lungs, hepatobiliary system, musculoskeletal system, gastrointestinal (GI) tract, and skin.

In vitro assessments have suggested that metabolism of belumosudil is primarily dependent on cytochrome P450 CYP3A4 activity and that the solubility of belumosudil is pH dependent. Preclinical investigations in mouse, rat, rabbit, and dog have indicated that belumosudil undergoes hepatic metabolism to form 2 main metabolites called KD025 ml (a ROCK2 active minor metabolite) and KD025m2 (a ROCK2 inactive major metabolite). These main metabolites were subsequently quantified in clinical trials across the belumosudil development program; exposure of the major, inactive metabolite KD025m2 was 15% to 20% of the parent, while the minor, active metabolite KDO25 ml had exposure value <5% of the parent.

In vitro assessments determined that cytochrome P450 CYP3A4 was the predominant CYP isoform responsible for belumosudil metabolism. Incubations of belumosudil with recombinant enzymes indicated that CYP3A4 was responsible for the metabolism of belumosudil (41.9%), although CYP2D6 (21.7%), CYP2C8 (14.2%), CYP1A2 (<5%), CYP2C19 (<5%), and uridine diphosphate glucuronosyltransferase 1Al may contribute to a lesser extent.

As such, a two-part clinical drug-drug interaction study was conducted to assess the effect of itraconazole (a CYP3A4 inhibitor), rifampicin (a CYP3A4 inducer), and rabeprazole, and omeprazole (both proton pump inhibitors [PPIs]) on the pharmacokinetics of belumosudil which is described in Example 1.

No clinically relevant change in belumosudil exposure was observed following a 200 mg single oral dose of belumosudil with itraconazole; however, exposure of main, inactive metabolite KD025m2 was decreased. Conversely, the CYP3A4 inducer rifampicin significantly decreased exposure of belumosudil and its inactive metabolite, KD025m2 and increased exposure to the active metabolite KD025 ml. Coadministration of rifampin may decrease belumosudil Cₘₐₓ by 59% and AUC by 72% in healthy subjects. Coadministration of efavirenz (a CYP3A Inducer) may decrease belumosudil Cₘₐₓ by 32% and AUC by 35% in healthy subjects.

When a 200 mg single oral dose of belumosudil was co-administered with PPIs rabeprazole and omeprazole, parent and metabolite exposures were largely reduced. Coadministration of rabeprazole may decrease belumosudil Cₘₐₓ by 87% and AUC by 80%, and omeprazole may decrease belumosudil Cₘₐₓ by 68% and AUC by 47% in healthy subjects.

Administration of belumosudil with and without perpetrator compounds was safe, and no notable adverse events were reported.

Accordingly, coadministration of belumosudil with CYP3A inducers decreases belumosudil exposure. The dosage of belumosudil should therefore be increased when co-administered with CYP3A inducers. For example, in one embodiment, the dosage of belumosudil is increased to 200 mg twice daily or 400 mg daily when co-administered with strong CYP3A inducers.

Coadministration of belumosudil with proton pump inhibitors (PPIs) also decreases belumosudil exposure. The dosage of belumosudil may be increased when co-administered with proton pump inhibitors. For example, in one embodiment, the dosage of belumosudil is increased to 200 mg twice daily or 400 mg daily when co-administered with proton pump inhibitors.

### Definitions

“About” as used herein includes the exact amount modified by the term, about, as wells as an amount that would be expected to be within experimental error, such as for example, within 15%, 10%, or 5%. For example, “about 200 mg” means “200 mg” and also a range of mgs that is within experimental error, e.g., plus or minus 15%, 10%, or 5% of 200 mg. As used herein, the term “about” may be used to modify a range and also, a particular value.

“Administering” or “administered to” as used herein (for example, with reference to administration of APIs, including “co-administration” of one or more APIs, such as Compound, belumosudil, PPIs, and/or CYP3A inducers to a subject), refers to the act of prescribing medicine(s) containing one or more of the APIs for the subject to take during treatment, the act of prescribing a protocol of medicines to be taken by a subject, the act of dispensing the medicine(s) to the subject, and/or the act of physically receiving or ingesting the medicine(s). Thus, the APIs (e.g., Compound, belumosudil, PPIs and/or CYP3A inducers), can be “administered” by a physician or other medical professional who writes prescriptions for any one of such medicine(s); and/or by a pharmacist who fills said prescriptions and/or dispenses one or more of the medicine(s) to the subject; and/or by the patient or subject who ingests the medicine(s) and/or his or her partner or caretaker who delivers the medicine(s) to the subject.

“API” means “active pharmaceutical ingredient.”

“Allogeneic hematopoietic stem cell transplantation (allo-HSCT)” also called bone marrow transplantation or stem cell transplantation, or “allogeneic hematopoietic cell transplantation (allo-HCT)” refers to a procedure where hematopoictic cells from a donor are grafted into a recipient who is not an identical twin. The source of hematopoietic stem cells for allogeneic transplantation may be peripheral blood stem cells (PBSC) or bone marrow (BM). In some circumstances umbilical cord blood may be used. The donor and recipient may be matched at the human leukocyte antigen (HLA) genes, such as siblings. The donor and recipient may be a parent and a child who are only half-matched (haploidentical).

Belumosudil is an oral selective rho-associated coiled-coil-containing protein kinase-2 (ROCK2) inhibitor. ROCK2 inhibition acts on the dysregulated adaptive immune system and the fibrosis that occurs as a result of aberrant tissue repair. Belumosudil inhibits ROCK2 and ROCK1 with ICso values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of chronic GVHD.

The compound belumosudil has the chemical name: 2-{3-[4-(1H-indazol-5-ylamino)-2-quinazolinyl] phenoxy}-N-(propan-2-yl) acetamide. The compound belumosudil is also known as KD025. The mesylate salt of belumosudil is marketed as REZUROCK™ in the United States for the treatment of patients with chronic GVHD after failure of at least two prior lines of systemic therapy. The active pharmaceutical ingredient of REZUROCK™ is belumosudil mesylate salt with the molecular formula C27H28N6O5S, a molecular weight of 548.62 g/mol, and having the chemical name 2-{3-[4-(1H-indazol-5-ylamino)-2-quinazolinyl] phenoxy}-N-(propan-2-yl) acetamide methanesulfonate (1:1).

The chemical structure of belumosudil mesylate is as follows:

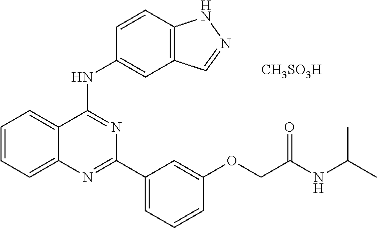

Belumosudil and processes for making the compound are described in the following US patents: U.S. Pat. Nos. 8,357,693, 9,815,820, 10,183,931, and 10,696,660.

When the term “Belumosudil” is used herein, it should be understood that, unless the context clearly indicates otherwise, the term may cover the compound belumosudil in any form as well as pharmaceutically acceptable salts thereof. The term “Belumosudil” refers both to the compound belumosudil (for example, in the free base form, amorphous form, or crystalline form), to pharmaceutically acceptable salts of belumosudil, for example, the mesylate salt form as used in as REZUROCK,™ and to any form of belumosudil that may be used in a formulation or pharmaceutical composition for administering the compound to a patient.

“Clinical endpoint” or “study endpoint” refers to an event or outcome in a clinical trial that can be measured objectively to determine outcomes and potential beneficial effects of the drug or administration protocol as designed in the clinical trial. Examples of clinical endpoints include the following. Overall response rate (ORR) is the percentage of people in a study or treatment group who have a partial response (PR) or complete response (CR) to the treatment within a certain period of time. Failure-free survival (FFS) means the time from the first dose of belumosudil to a failure event, or the interval between the start of belumosudil and the addition of a new cGVHD therapy, relapse of the underlying disease, or nonrelapse mortality (NRM). Overall survival (OS) means the length of time from either the date of diagnosis or the start of treatment for a disease. Duration of response (DOR) means from the time of initial response (e.g., PR or CR) until documented progression from best response of cGVHD, time from initial response to start of additional systemic cGVHD therapy, or death. Time to next treatment (TTNT) means time to initiation of a subsequent systemic cGVHD therapy.

“Clinically recommended amount” or “clinically recommended dosage” refers to the amount or dosage of API that has been recommended and/or approved for administration to a subject by those skilled in the field of medicinal chemistry to treat the disease state in question following clinical trials, for example, as set forth in publications, clinical trial results, and on the approved drug label. In one embodiment, a clinically recommended dosage for belumosudil, without administration of CYP3A inducers or PPIs, as indicated on the drug label for belumosudil, is 200 mg once daily.

“Co-administration,” “in combination with,” and/or “co-administered,” as used herein with reference to administration of Belumosudil with CYP3A Inducers and/or PPIs means that during the course of the patient's treatment with Belumosudil, the patient is also receiving one or more dosages of one or more CYP3A Inducers and/or PPIs (also referred to herein as “perpetrator compounds”). The perpetrator compounds need not be administered concomitantly with, or on the same day, as the Belumosudil to be considered as being “co-administered” under this definition. The perpetrator compounds may be administered at the same time as the Belumosudil, for a number of days prior to the administration of Belumosudil, and/or during the course of treatment to be “co-administered.” As described in Example 1, a perpetrator compound will not be considered “co-administered” or administered “in combination with” the Belumosudil if there is a sufficient washout period between administrations to account for a minimum of 5 half-lives of the active moieties dosed.

“Compound” as used in the Claims and Embodiments herein, and when apparent from context of usage, is synonymous with the above all-inclusive definition of Belumosudil.

“Concurrent administration,” “concurrently” and/or “concurrently taking” with reference to administration of APIs herein (e.g., as applied to CYP3A inducers and/or PPIs “concurrently” administered with belumosudil), is synonymous with “co-administered,” as defined above.

“CYP3A” refers to the CYP3A family of p-450 isoenzymes including CYP3A4. Examples of CYP3A inducers may include glucocorticoids, carbamazepine, apalutamide, enzalutamide, mitotane, phenytoin, rifampin (rifamicin), fosphenytoin, lumacaftor, lumacaftor-ivacaftor, mitotane, and St. John's Wort. A CYP3A inducer may include phenobarbital, bosentan, efavirenz, etravirine, primidone, bexarotene, cenobamate, dabrafenib, dexamethasone, dipyrone, elagolix, estradiol, eslicarbazepine, lorlatinib, mitapivat, modafinil, nafcillin, pexidartinib, rifabutin, rifapentine, and sotorasib. Additional CYP3A inducers may include armodafinil, modafinil, and rufinamide. In some embodiments herein, the CYP3A inducers are considered strong CYP3A inducers. Examples of strong CYP3A inducers include rifampicin and phenytoin.

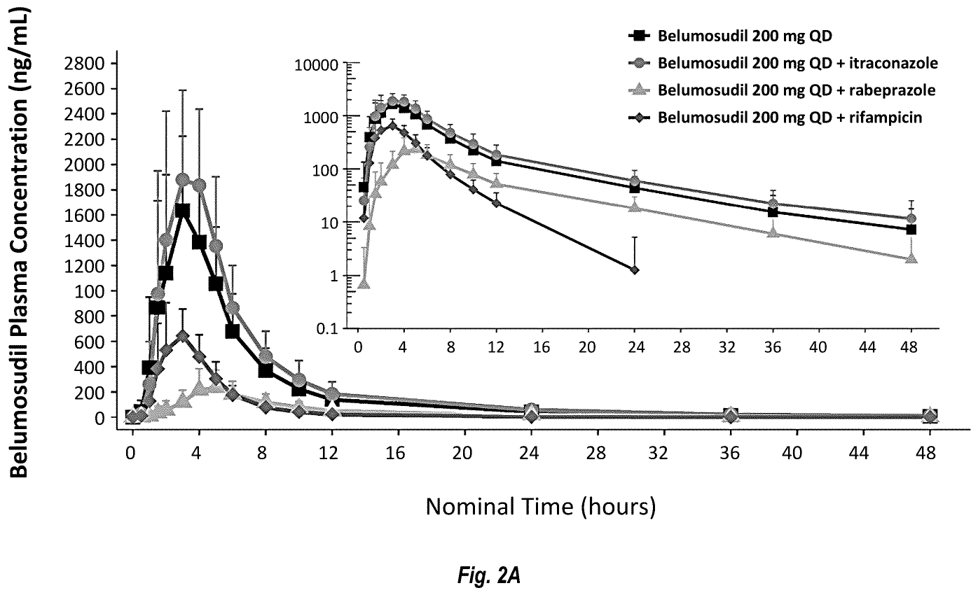

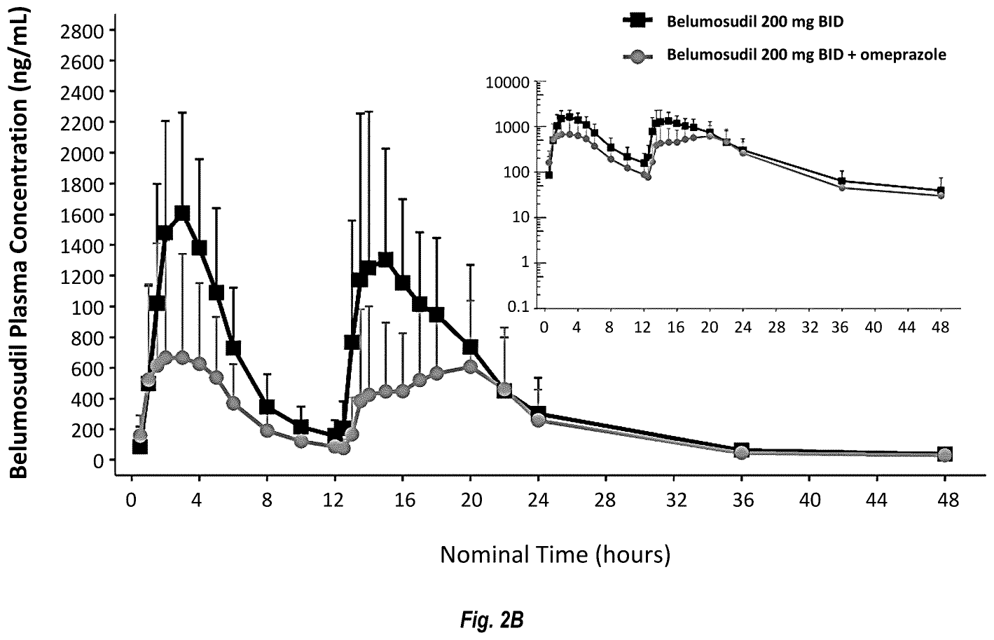

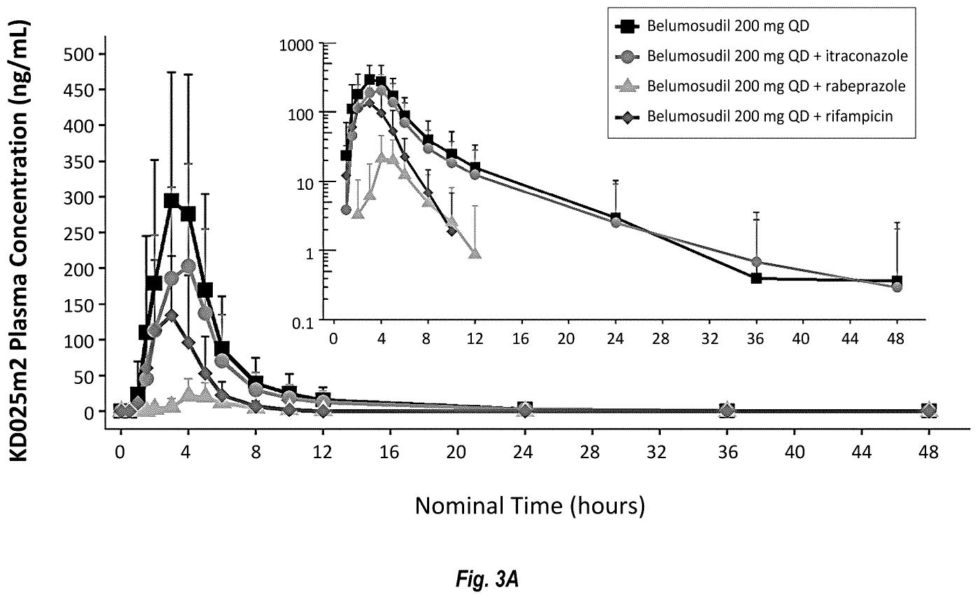

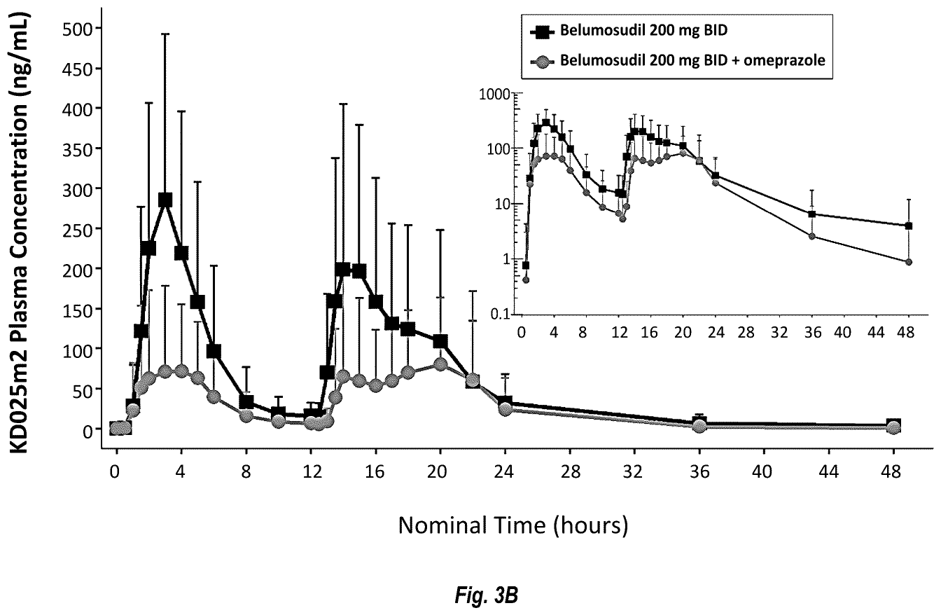

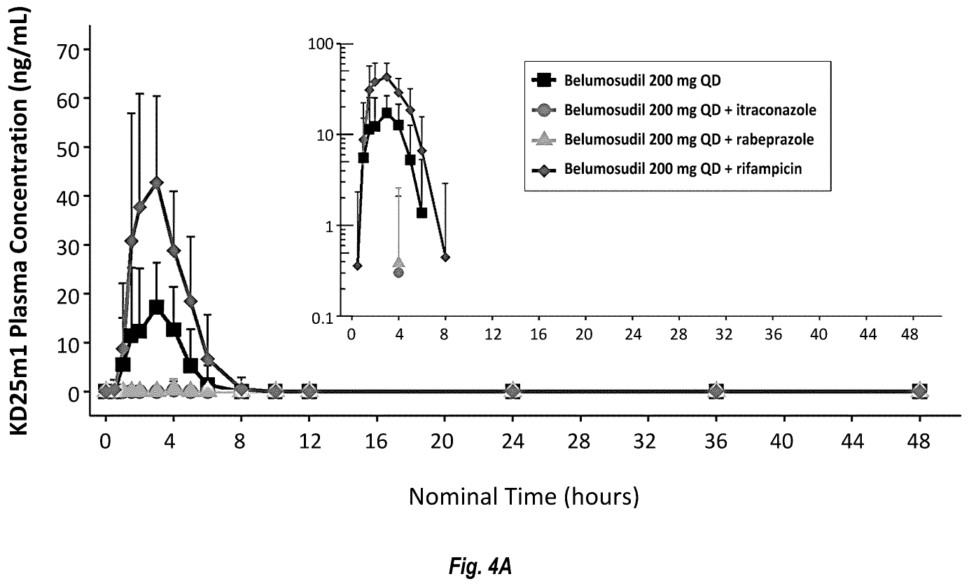

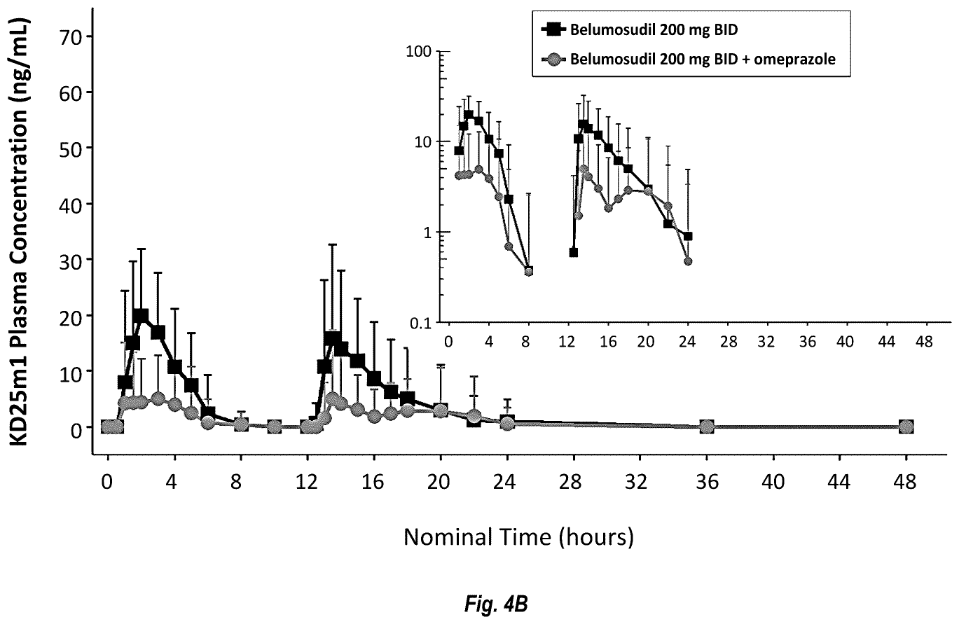

“Exposure reduction effect” or “exposure reduction effects” as used herein refers to the impact of CYP3A and PPI compounds on belumosudil PK, for example, exposure levels (overall and peak) for belumosudil and its metabolites and rates of elimination of belumosudil and its major metabolites, KDO25 ml and KD025m2. Reference is further made to Example 1 and FIGS. 2A-FIG. 4B hereof as exemplification of the term “exposure reduction effect.”

A high-fat, high-calorie meal, as used herein, means a meal containing about 800 to 1,000 calories with approximately 50% of total caloric content of the meal from fat food. For example, in one embodiment, a high-fat breakfast may consist of hash browns, bacon, fried egg, white bread, and 240 mL of full fat milk.

Lec Symptom Scale (LSS) summary score measures the effect on patients' functioning and well-being. The Lee Symptom Scale is a 30-item scale developed to measure the symptoms of cGVHD and is described in Lec SJ, et al., Development and validation of a scale to measure symptoms of chronic graft-versus host disease. Biol Blood Marrow Transplant 2002; 8:444-452.

“Line of treatment” or “line of therapy” describes the sequence or order in which different therapies are given to a patient as the patient's disease progresses. Initial treatment (first-line therapy) may not work or may stop working after a period. After first-line therapy is discontinued, a second different treatment (second-line therapy) may be given. Subsequent lines of therapy may be given when a second-line therapy does not work or stops working. Some patients may be administered multiple lines of therapy over the course of a disease.

First-line therapy for National Institutes of Health (NIH)-defined moderate to severe chronic graft-versus-host disease (cGVHD) may be corticosteroids alone or in combination with sirolimus or a calcineurin inhibitor. (Carpenter PA, et al.: A phase II/III randomized, multicenter trial of prednisone/sirolimus versus prednisone/sirolimus/calcineurin inhibitor for the treatment of chronic graft-versus-host disease: BMT CTN 0801. Haematologica 103:1915-1924, 2018).

Examples of corticosteroid therapies for treatment of cGVHD include, but are not limited to, prednisone, prednisolone, methylprednisolone, and budesonide. Examples of prior systemic therapies for treating cGVHD include, but are not limited to, prednisone, tacrolimus, extracorporeal photopheresis (ECP), sirolimus, ibruitinib, ruxolitinib, mycophenolate mofetil (MMF), rituximab, methotrexate (MTX), cyclosporine, imatinib, ixazomib, and ofatumumab.

“Immunosuppressive therapy” (IST) refers to therapy that is typically administered for at least six months after allo-HSCT to try to prevent GVHD. Examples of IST's include sirolimus, prednisone and calcineurin inhibitors such as tacrolimus and cyclosporine.

“Myeloablative transplant” refers to a transplantation process using very high doses of chemotherapy or radiation prior to transplantation with autologous or allogeneic hematopoietic stem cells. A non-myeloablative transplant, or reduced intensity transplant, involves the patient having less intensive chemotherapy before transplantation with allogeneic hematopoietic stem cells.

“NIH lung symptom score” or “NIH cGVHD lung score” is a clinical symptom-based score ranging from 0 to 3. A Score 0 is used for no symptoms, Score 1 is used for symptoms of shortness of breath with stairs, Score 2 is used for symptoms of shortness of breath on flat ground, and Score 3 is used for shortness of breath at rest or requiring oxygen.

“Or” is used in the inclusive sense (equivalent to “and/or”) unless the context requires otherwise.

“Patient” or “subject” as used herein includes an animal or a human; in one embodiment, the term “patient” refers to a human subject.

“Perpetrator compound” as used herein refers to a compound investigated for its co-administration effects, for example, CYP3A Inducers and PPIs.

“Protocol” as used herein refers to the methods or plan that is used to administer one or more APIs to a subject in need of treatment. The term “protocol” is intended to encompass the overall, detailed plan of care for a patient, as well as individual or partial steps that are part of the overall plan. For example, a protocol may include the dosages used for each API the patient will be (or is) receiving, the combination of APIs the patient receives, the timing and method of administration of each API (for example, considering DDIs, food effects, and impact different formulations or modes of delivery may have on absorption and bioavailability), and management of side effects, as well as the overall plan encompassing the dosages, combinations, timing and methods of administration, and side effects, considered together.

“Proton pump inhibitor” or “PPI” refers to a drug that inhibits the stomach's H+/K+ATPase proton pump and causes a reduction in stomach acid production. As PPIs reduce stomach acid production, they can increase the pH of the stomach which impact the solubility and potentially the bioavailability of orally delivered medicines. Examples of PPIs include omeprazole, lansoprazole, dexlansoprazole, esomeprazole, pantoprazole, rabeprazole, and ilaprazole.

“Steroid-refractory” (SR) cGVHD is defined as cGVHD progression while on steroids or corticosteroids; in one embodiment, while on prednisone.

“Standard treatment conditions” refers to treatment and/or dosage regimes for administration of Belumosudil to a patient for treatment of cGVHD wherein a CYP3A inducer or PPI is not also co-administered to the patient during the course of treatment, pursuant to the above definition of “co-administered.”

A “therapeutically effective amount” of an API means an amount which, when administered to a human for treating a disease (for example, cGVHD), is sufficient to effect treatment for the disease state being treated. As applied to cGVHD in a human, “treating” or “treatment” includes (1) reducing the risk of developing cGVHD and/or inhibiting cGVHD, i.e., arresting or reducing the development of cGVHD or its clinical symptoms; and (2) relieving cGVHD, i.e., causing regression, reversal, or amelioration of the cGVHD or reducing the number, frequency, duration or severity of its clinical symptoms.

The therapeutically effective amount of an API may vary depending upon the health and physical condition of the subject to be treated, the extent of disease progression, the assessment of the medical situation, and other relevant factors. It is expected that the therapeutically effective amount may fall within a range that can be determined through trial and through reference to clinical trial data and results, for example, as described in Examples 1 and 3 hereof and in scientific literature.

### Exemplary Embodiments

In some embodiments, there is provided belumosudil or a pharmaceutically acceptable salt thereof (Compound) for use in the treatment of chronic graft-versus-host-disease (cGVHD) in a subject, wherein at least one CYP3A Inducer or Proton Pump Inhibitor (PPI) is co-administered to the subject.

In another embodiment, at least one CYP3A Inducer is co-administered to the subject, concurrently with belumosudil; in another embodiment, the CYP3A Inducer is a glucocorticoid, carbamazepine, apalutamide, enzalutamide, mitotane, phenytoin, rifampin (rifampicin), fosphenytoin, lumacaftor, lumacaftor-ivacaftor, mitotane, St. John's Wort, phenobarbital, bosentan, efavirenz, etravirine, primidone, bexarotene, cenobamate, dabrafenib, dexamethasone, dipyrone, elagolix, estradiol, eslicarbazepine, lorlatinib, mitapivat, modafinil, nafcillin, pexidartinib, rifabutin, rifapentine, sotorasib, armodafinil, modafinil, or rufinamide. In another embodiment, the CYP3A Inducer is a CYP3A4 inducer.

In another embodiment, the CYP3A inducer is a strong CYP3A inducer; in one embodiment, the strong CYP3A inducer is rifampicin or phenytoin.

In another embodiment, the CYP3A4 inducer is phenobarbital, phenytoin, rifampicin, St. John's Wort or a glucocorticoid.

In another embodiment, the CYP3A4 inducer is rifampicin.

In another embodiment, a PPI is co-administered to the subject concurrently with belumosudil.

In another embodiment, the PPI is omeprazole, lansoprazole, dexlansoprazole, esomeprazole, pantoprazole, rabeprazole, or ilaprazole.

In another embodiment, the PPI is rabeprazole or omeprazole.

In another embodiment, the dose of the belumosudil according to the co-administration disclosed herein is about 400 mg daily; in other embodiments, the dose of the Compound (belumosudil) is about 200 mg administered twice daily for a total daily dose of about 400 mg; in other embodiments, the dose of the Compound (belumosudil), is in the range of about 400 mg to 800 mg daily; in other embodiments, the dose of Compound (belumosudil), is 400 mg daily, 400 mg twice daily, or 800 mg daily.

In some embodiments, the dose of the Compound is increased over the clinically recommended dose of the Compound for the subject under standard treatment conditions to mediate exposure reduction effects of a co-administered CYP3A Inducer and/or PPI.

In another embodiment, there is provided a method of co-administering the CYP3A Inducer rifampicin with belumosudil, wherein the exposure reduction effect of rifampicin is characterized by a decrease in belumosudil Cₘₐₓ by about 59% and/or AUC by about 72%.

In another embodiment, there is provided a method of co-administering the CYP3A Inducer efavirenz with belumosudil, and the exposure reduction effect of efavirenz is characterized by a decrease in belumosudil Cₘₐₓ by about 32% and/or AUC by about 35%.

In another embodiment, there is provided a method of co-administering a PPI (e.g., omeprazole, rabeprazole) with belumosudil, where there are exposure reduction effects characterized by a decrease in belumosudil Cₘₐₓ and/or AUC as substantially shown in Table 4 herein.

In another embodiment, there is provided a method of co-administering a PPI and/or CYP3A inducer with belumosudil, wherein the CYP3A inducer is rifampicin administered at a daily dose dependent upon the body weight of the subject and in the range of from about 10 to 20 mg/kilogram; and/or wherein the CYP3A inducer is rifampicin administered at a dose of about 600 mg daily for about 5 to 9 days; or wherein the PPI is omeprazole administered at a dose of about 20 mg to 120 mg daily; or wherein the PPI is omeprazole administered at a dose of 20 mg daily; or wherein the PPI is rabeprazole administered at a dose of about 5 to 40 mg daily; or wherein the PPI is rabeprazole administered at a daily dose of 20 mg given twice daily.

In some embodiments, the use or methods comprise a treatment cycle wherein the CYP3A inducer or PPI is administered sequentially on days on which the subject does not receive a dose of the Compound (belumosudil); in some embodiments, the CYP3A inducer or PPI is administered prior to administration of the Compound.

In some embodiments, the use or methods comprise a treatment cycle wherein the CYP3A inducer or PPI is administered substantially at the same time as the dose of the Compound (belumosudil).

In some embodiments, there is provided a method of administering Compound (or belumosudil), to a subject comprising administering a high-fat and high-calorie meal within about one hour or less prior to administration of the Compound; in some embodiments, within about thirty minutes or less; in some embodiments, between thirty minutes and one hour before Compound administration.

In some embodiments, there is provided a method of treating chronic graft-versus-host-disease (cGVHD) in a subject who is concurrently taking a CYP3A Inducer or Proton Pump Inhibitor (PPI) comprising administering to the subject in need thereof a therapeutically effective amount of belumosudil mesylate salt.

In some embodiments, there is provided a method for treating a subject with cGVHD comprising the steps of:
a) determining whether the subject is in need of treatment with at least one CYP3A Inducer or PPI, and if so, the dose thereof;

b) calculating the therapeutically-effective dose of 2-{3-[4-(1H-indazol-5-ylamino)-2-quinazolinyl phenoxy}-N- (propan-2-yl) acetamide, or pharmaceutically acceptable salts thereof (Compound), for the subject under standard conditions; and
c) determining an upward adjusted dose of Compound to be administered to the subject to mediate the exposure reduction effect of the CYP3A Inducer and/or Proton Pump Inhibitor in treating the cGVHD.

In some embodiments, there is provided a method of administering an adjusted dose of belumosudil to account for exposure reduction effects of a PPI and/or CYP3A inducer, wherein the exposure reduction effect is characterized by a decrease in belumosudil Cₘₐₓ by about 30% to 90%; in some embodiments, the exposure reduction effect is characterized by a decrease in belumosudil Cₘₐₓ by about 30% to 80%; in some embodiments, the exposure reduction effect is characterized by a decrease in belumosudil Cₘₐₓ by about 35% to 75%; in some embodiments, the exposure reduction effect is characterized by a decrease in belumosudil Cₘₐₓ by about 55% to 90%; in some embodiments, the exposure reduction effect is characterized by a decrease in AUC by about 60% to 90%; in some embodiments, the exposure reduction effect is characterized by a decrease in belumosudil AUC by about 55% to 85%; in some embodiments, the exposure reduction effect is characterized by a decrease in belumosudil AUC by about 40% to 60%.

In some embodiments, there is provided a method of treating chronic graft-versus-host-disease (cGVHD) in a subject who is concurrently taking a CYP3A Inducer or Proton Pump Inhibitor (PPI) comprising administering to the subject in need thereof a protocol to mediate the exposure reduction effects of the CYP3A Inducer or PPI and taking into consideration the food effect.

In some embodiments, the subject receiving said treatment has had allogeneic hematopoietic stem cell transplantation that is a matched-HSCT. In some embodiments, the allogeneic hematopoietic stem cell transplantation is a haploidentical-HSCT.

In some embodiments, the belumosudil treatment is continued based on the patient's tolerability until active cGVHD symptoms resolve or progress. The number of cycles and duration of the treatment is patient dependent. In some embodiments, the belumosudil is administered to the patient in one or more 28-day cycles.

In some embodiments, the number of cycles ranges from 3 to 15. In some embodiments, the number of cycles ranges from 3 to 14, from 3 to 13, from 3 to 12, from 3 to 11, from 3 to 10, from 3 to 9, from 3 to 8, from 3 to 7, from 3 to 6, from 3 to 5, or from 3 to 4. In some embodiments, the number of cycles ranges from 5 to 11. In some embodiments, the number of cycles ranges from 6 to 12. In some embodiments, the number of cycles ranges from 5 to 10, from 5 to 9, or from 5 to 8. In some embodiments, the number of cycles ranges from 5 to 7. In some embodiments, the number of cycles ranges from 5 to 6. In some embodiments, the number of cycles is 5. In some embodiments, the number of cycles is 6. In some embodiments, the number of cycles is 7. In some embodiments, the number of cycles is 3, 4, 5, 6, 7, 8, 9, 10, 11, 12, 13, 14, or 15.

In some embodiments, the number of cycles ranges from 3 cycles to loss of response. In some embodiments, the number of cycles ranges from 4 cycles to loss of response. In some embodiments, the number of cycles ranges from 5 cycles to loss of response. In some embodiments, the number of cycles ranges from 6 cycles to loss of response. In some embodiments, the number of cycles ranges from 7 cycles to loss of response. In some embodiments, the number of cycles ranges from 8 cycles to loss of response. In some embodiments, the number of cycles is greater than 3, 4, 5, 10, 15, 20, 25, or 30, or until a desired response is achieved.

In some embodiments, the subject experiences an improvement as defined by the Lec Symptom Scale (LSS). In some embodiments, the subject experiences at least a 7-point reduction in the LSS score. In some embodiments, the subject experiences at least a 10-point reduction in the LSS score. In some embodiments, the improvement is maintained over at least two consecutive evaluations. In some embodiments the LSS score is evaluated at baseline and on day 1 of each cycle starting at cycle 2 day 1.

In some embodiments, the subject has chronic graft-versus-host disease and has failed one to three prior lines of systemic therapy for the chronic graft-versus-host disease. In some embodiments, the subject has chronic graft-versus-host disease and has failed at least two prior lines of systemic therapy for the chronic graft-versus-host disease. In some embodiments, the subject has chronic graft-versus-host disease and has failed two to five prior lines of systemic therapy for the chronic graft-versus-host disease. In some embodiments, the subject has failed at least one, at least two, at least three, at least four, or at least five prior lines of systemic therapy for the chronic graft-versus-host disease.

In some embodiments, the subject experienced a complete response to last treatment for the graft-versus-host disease prior to belumosudil. In some embodiments, the subject experienced a partial response to last treatment for the graft-versus-host disease prior to belumosudil. In some embodiments, stable disease during the last treatment for the graft-versus-host disease prior to belumosudil.

In some embodiments, the prior lines of systemic therapy for the chronic graft-versus-host disease have been discontinued.

In some embodiments, the prior lines of systemic therapy are selected from the group consisting of prednisone, tacrolimus, ECP, sirolimus, ibruitinib, ruxolitinib, MMF, rituximab, MTX, cyclosporine, imatinib, ixazomib, and ofatumumab.

In some embodiments, the cGVHD is steroid-refractory (SR) cGVHD. In some embodiments, the subject is refractory to the last line of treatment prior to belumosudil treatment.

In some embodiments, the subject is receiving concomitant corticosteroid therapy. In some embodiments, the concomitant corticosteroid therapy is selected from the group consisting of prednisone, prednisolone, methylprednisolone, and budesonide. In some embodiments, the concomitant corticosteroid therapy is prednisone. In some embodiments, the dose of the concomitant corticosteroid therapy is reduced after at least 1 cycle of the belumosudil treatment. In some embodiments, the dose of the concomitant corticosteroid therapy is reduced by at least about 10%, by at least about 20%, by at least about 30%, by at least about 40%, by at least about 50%, by at least about 60%, or by at least about 70% after at least 1 cycle of the belumosudil treatment. In some embodiments, the dose of the concomitant corticosteroid therapy is reduced by from about 10% to about 70%, from about 15% to about 65%, from about 20% to about 60%, from about 30% to about 60%, from about 35% to about 60%, from about 40% to about 60%, or from about 45% to about 55% after at least 1 cycle of the belumosudil treatment. In some embodiments, the concomitant corticosteroid therapy is discontinued after at least 1 cycle of the belumosudil treatment.

In some embodiments, the subject is receiving concomitant calcineurin inhibitor therapy.

In some embodiments, the subject has involvement of at least 4 organs. In some embodiments, the subject has involvement of at least 3 organs. In some embodiments, the subject has involvement of at least 2 organs.

### Tablets

Belumosudil mesylate is a yellow powder that is practically insoluble in water. Belumosudil tablets may be prepared for oral administration. Each tablet contains 200 mg of the free base equivalent to 242.5 mg of belumosudil mesylate. The tablet also may contain the following inactive ingredients: microcrystalline cellulose, hypromellose, croscarmellose sodium, colloidal silicon dioxide, and magnesium stearate. The tablet film consists of polyvinyl alcohol, polyethylene glycol, talc, titanium dioxide and yellow iron oxide. Each 200 mg tablet is a pale yellow film-coated oblong tablet debossed with “KDM” on one side and “200” on the other side. Tablets are stored at room temperature, 20° C. to 25° C. (68° F. to 77° F.); excursions permitted from 15° C. and 30° C. (59° F. to 86° F.).

### Clinical Approval and Pharmacokinetics

Based on the efficacy and safety observed in clinical studies, when belumosudil is administered without co-administration of CYP3A Inducers and/or Proton Pump Inhibitors, 200 mg daily was selected as the preferred dosage for treatment of SR cGVHD. The approved drug label for belumosudil mesylate salt (REZUROCK™) states that the recommended dosage is 200 mg taken orally once daily with food.

Use of a 200-mg twice-daily dose was well tolerated and showed higher responses in certain organs, such as the skin. However, the difference compared with the 200-mg daily dose was not deemed significant in those settings for labeling purposes.

The following pharmacokinetic parameters are presented below for chronic GVHD patients administered belumosudil 200 mg once daily, unless otherwise specified. The mean (% coefficient of variation, % CV) steady-state AUC and Cₘₐₓ of belumosudil was 22700 (48%) hong/mL and 2390 (44%) ng/ml, respectively. Belumosudil Cₘₐₓ and AUC increased in an approximately proportional manner over a dosage range of 200 and 400 mg (1 to 2 times once daily recommended dosage). The accumulation ratio of belumosudil was 1.4.

Absorption: Median Tₘₐₓ of belumosudil at steady state was 1.26 to 2.53 hours following administration of 200 mg once daily or twice daily in patients. The mean (% CV) bioavailability was 64% (17%) following a single belumosudil dose in healthy subjects.

Food Effect: Belumosudil Cₘₐₓ and AUC increased 2.2 times and 2 times, respectively, following administration of a single belumosudil dose with a high-fat and high-calorie meal (800 to 1,000 calories with approximately 50% of total caloric content of the meal from fat) compared to the fasted state in healthy subjects. Median Tₘₐₓ was delayed 0.5 hours.

Distribution: The geometric mean volume of distribution after a single dose of belumosudil in healthy subjects was 184 L (geo CV % 67.7%). Belumosudil binding to human serum albumin and human al-acid glycoprotein was 99.9% and 98.6%, respectively, in vitro.

Elimination, Metabolism, and Excretion: The mean (% CV) elimination half-life of belumosudil was 19 hours (39%), and clearance was 9.83 L/hours (46%) in patients. Belumosudil is primarily metabolized by CYP3A4 and to a lesser extent by CYP2C8, CYP2D6, and UGT1A9, in vitro. Following a single oral dose of radiolabeled belumosudil in healthy subjects, 85% of radioactivity was recovered in feces (30% as unchanged) and less than 5% in urine.

The following abbreviations may be helpful in considering the Examples and description herein.

### Abbreviations

AE Adverse events
alloHCT allogeneic hematopoietic cell transplantation
BID Twice daily (bi-daily)
BM Bone marrow
cGVHD Chronic graft versus host disease
CMV cytomegalovirus
CR Complete response
DDI Drug-drug interaction
DOR Duration of response
FFS Failure-free survival
HLA human leukocyte antigen
IST Immunosuppressive therapy
LSS Lee Symptom Scale
ORR Overall response rate
OS Overall survival
PBSC peripheral blood stem cells
PPI Proton pump inhibitors
PR Partial response
QOL Quality of life
SD Standard deviation
SR Steroid refractory
TEAEs treatment-emergent adverse events
TTNT Time to next treatment
QD Daily; every day

### Examples

Example 1: A phase I, two-part, open label study to evaluate the effect of itraconazole, rifampicin, rabeprazole and omeprazole on the pharmacokinetics of belumosudil

A clinical drug-drug interaction (DDI) study was conducted to investigate the effect of CYP3A4 inhibitors and CYP3A4 inducers on belumosudil pharmacokinetics (PK). Itraconazole and rifampicin, a CYP3A4 inhibitor and inducer, respectively, were selected as exemplary DDI candidates for this assessment. Additionally, because the solubility of belumosudil is pH dependent (i.e., 0.011, 1.644, 1.433, 0.003, and 0.000 mg/mL at pH levels of 1.08, 1.6, 2.0, 6.5, and 6.8, respectively), interactions between proton pump inhibitors (PPIs) and belumosudil were also assessed in this DDI study.

The primary objectives of this DDI study were: (1) to determine the effect of itraconazole, rifampicin, and rabeprazole on the PK of belumosudil following a single oral 200 mg dose, and (2) to determine the effect of omeprazole on the PK of belumosudil following a 200 mg twice daily (BID) dose, in healthy male subjects. The secondary objective was to provide additional information on the safety and tolerability of this dose regimen in healthy males.

### Study Design

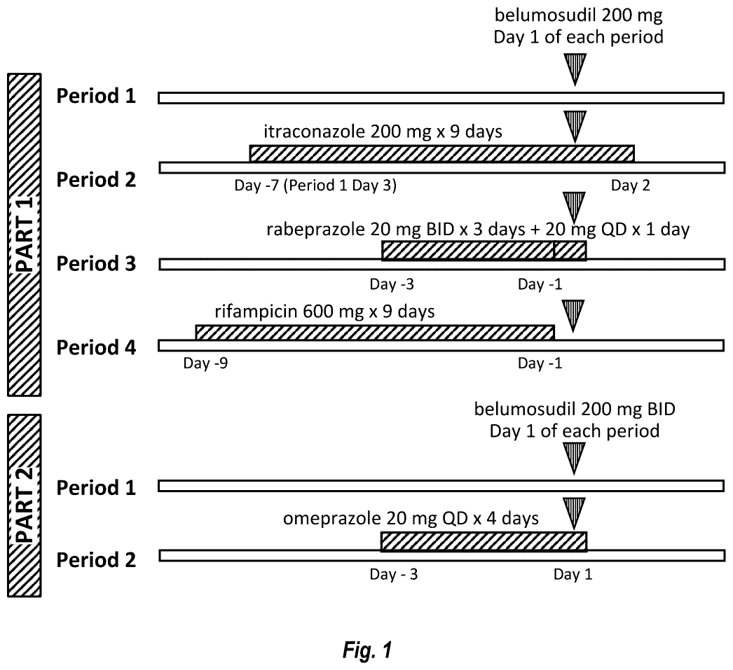

This was a single center, non-randomized, DDI study in 35 (Part 1) and 38 (Part 2) healthy males. The key inclusion criterion was males aged 18 to 55 years (inclusive). The study was conducted in 2 parts: in Part 1 (Periods 1 through 4), belumosudil was investigated when co-administered with itraconazole, rabeprazole, and rifampicin. In Part 2, co-administration with omeprazole was studied. The study design is illustrated in FIG. 1.

On Day 1 of each period in Part 1 (Periods 1 to 4), 35 subjects received a single dose of 200 mg belumosudil tablet. In Periods 2 to 4, repeat doses of 200 mg itraconazole QD, 20 mg rabeprazole BID, or 600 mg rifampicin QD were also administered for a number of days prior to belumosudil dosing and concomitantly with belumosudil on Day 1 (itraconazole and rabeprazole only). At least a 2-to 8-day washout occurred between each period to account for a minimum of 5 half-lives of the active moieties dosed in the preceding period. On Day 1 of each period in Part 2 (Periods 1 and 2), 38 separate subjects received 200 mg belumosudil tablet BID (12 hours apart). During Period 2, repeat doses of 20 mg omeprazole QD were given for 3 days prior to belumosudil dosing and with the Day 1 belumosudil dosc.

In both study parts, subjects were fasted overnight before belumosudil dosing. Prior to receiving belumosudil, a standard breakfast was provided, consisting of 1 bowl of cereal, 200 mL semi-skimmed milk, and 1 croissant or roll with pre-packaged jam. All belumosudil doses were administered 30 minutes after the start of the meal, as administration of belumosudil with food decreases the rate and increases the extent of absorption as described in Example 2.

### Sample Collection and Analysis

In each period of Parts 1 and 2, blood was collected for analysis of belumosudil, KD025 ml, and KD025m2 at predose and 0.5, 1, 1.5, 2, 3, 4, 5, 6, 8, 10, 12, 24, 36, and 48 hours post-belumosudil dose. Additional blood samples were collected in Part 2 at 12.5, 13, 13.5, 14, 15, 16, 17, 18, 20, and 22 hours post-belumosudil dose. Blood samples were collected into 6 mL K,EDTA tubes and were processed within 30 minutes of collection. The resultant plasma was then frozen within 90 minutes of collection and stored at 70° C. until shipment to the bioanalytical laboratory.

Belumosudil and its 2 metabolites (KD025 ml and KD025m2) were prepared for sample analysis using a protein precipitation method and were quantified by a liquid chromatography with tandem mass spectrometry (LC-MS/MS) method validated from 10 to 5000 ng/ml. While perpetrator compound concentrations were not quantified, co-administration studies were conducted to demonstrate that the presence of itraconazole, rabeprazole, rifampicin, and omeprazole does not affect the quantification of belumosudil in human plasma.

### Statistical Data Analysis

Sample size calculations were based on the following assumptions: 1) intra-subject variability of 50% for Cₘₐₓ and 40% for AUCₗₐₛₜ, based on the food effect; 2) 90% confidence interval (CI) for Cₘₐₓ and AUCₗₐₛₜ with Type 1 error of 0.05; 3) acceptance interval of 70.00 to 143.00%; and 4) 80% powder, assuming the true ratio is between 95.00 and 105.00. Based on these assumptions, it was estimated that 34 subjects evaluable for Cₘₐₓ, the PK parameter with the highest variability, were needed.

The PK population was defined as subjects who received at least 1 dose of belumosudil and had at least 1 valid post-dose concentration for PK parameter estimation. Additionally, subjects had to satisfy the following criteria for at least 1 analyte profile: 1) no missing samples at critical time points (e.g., around Cₘₐₓ), 2) no protocol deviations that would impact the study objectives with respect to the PK endpoints, and 3) no relevant AEs that suggest the full dose was not absorbed, such as vomiting. PK analysis datasets were a subset of the relevant PK population and included subjects with sufficient valid PK profiles who had completed both test (belumosudil+perpetrator compound) and reference (belumosudil alone) treatment periods to allow for relevant treatment comparisons.

PK parameters for plasma belumosudil, KDO25 ml, and KD025m2 were calculated using standard non-compartmental methods in Phoenix WinNonlin (v8.0). The primary PK parameters were Cₘₐₓ, AUCₗₐₛₜ, and AUC_{inf} for belumosudil and its main metabolites.

To evaluate the effect of each perpetrator compound on belumosudil PK (primary endpoint of the study), formal statistical analyses were conducted on Cₘₐₓ, AUCₗₐₛₜ, and AUC_{inf}. Pairwise treatment comparisons were made, with belumosudil 200 mg QD alone as the reference, and belumosudil 200 mg QD+perpetrator compound (itraconazole, rifampicin, rabeprazole, or omeprazole) as the test. For each comparison, PK parameters were natural log transformed and analyzed using mixed effect modeling with treatment included as a fixed effect and subject as a random effect. Adjusted geometric mean ratios (GMRs) and corresponding 90% CIs were calculated. The absence of an effect of the perpetrator compound on belumosudil PK was concluded if the 90% CIs for belumosudil Cₘₐₓ, AUCₗₐₛₜ, and AUC_{inf} were within the acceptance interval of 70.00 to 143.00%.

### Safety

Safety and tolerability of belumosudil was the secondary endpoint of the study and was evaluated by AEs, vital signs, physical examinations, electrocardiograms (ECGs), and clinical laboratory tests. All subjects who received at least one dose of belumosudil or perpetrator compound were included in the safety population.

### Results

Subject Disposition and Demographics

A total of 35 healthy males were enrolled and were dosed in Part 1 of the study. Five subjects discontinued early due to withdrawal of consent or discretion of the investigator (poor protocol compliance/positive drug test) and did not receive all 4 treatments. In Part 2 of the study, a total of 38 healthy males were enrolled and completed both treatment periods. Table 1 further illustrates the subject disposition for the study.

TABLE 1
Subject Disposition for Study of Example 1

| Disposition | Overall n (%) | |
|---|---|---|
| Subjects enrolled | | |
| Part 1 | 35 | |
| Part 2 | 38 | |
| Subjects dosed, Part 1 (total) | 35 | |
| Belumosudil alone | 35 | (100%) |
| Belumosudil + itraconazole | 35 | (100%) |
| Belumosudil + rabeprazole | 33 | (94.0%) |
| Belumosudil + rifampicin | 32 | (91.0%) |
| Subjects dosed, Part 2 (total) | 38 | |
| Belumosudil alone | 38 | (100%) |
| Belumosudil + omeprazole | 38 | (100%) |
| Subjects completed | | |
| Part 1 | 30 | (86.0%) |
| Part 2 | 38 | (100%) |
| Subjects discontinued, Part 1 | 5 | (14.0%) |
| Withdrawal by subject | 3 | (60.0%) |
| Physician decision | 2 | (40.0%) |

| | |
|---|---|
| Subjects discontinued, Part 2 | 0 |

Subjects' ages ranged from 20 to 55 years in Part 1 and 18 to 52 years in Part 2, with median ages of approximately 34 and 30 years, respectively. All subjects were within the protocol-defined body mass index (BMI) range, spanning from of 21.1 to 32.2 kg/m.2 The majority of subjects were white. Per study exclusion criteria, no study participants were current smokers, and all had an alcohol consumption between 0 and 20 units per week. The subject demographics for the study are further illustrated in Table 2.

TABLE 2
Subject Demographics by Treatment

| | | Treatment | | | | | | | | | |
|---|---|---|---|---|---|---|---|---|---|---|---|
| | | Belumosudil | | Belumosudil + | | Belumosudil + | | Belumosudil + | | Belumosudil + | |
| | | Alone | | Itraconazole | | Rabeprazole | | Rifampicin | | Omeprazole | |
| Demographic | Statistic | (N = 35) | | (N = 35) | | (N = 33) | | (N = 32) | | (N = 38) | |
| Age | Mean | 37.0 | (11.8) | 37.0 | (11.8) | 36.8 | (11.8) | 37.2 | (11.8) | 32.6 | (10.6) |
| (years) | (SD) | | | | | | | | | | |
| Sex | Male, n (%) | 35 | (100) | 35 | (100) | 33 | (100) | 32 | (100) | 38 | (100) |
| Race | Asian, n | 2 | (5.7) | 2 | (5.7) | 2 | (6.1) | 2 | (6.3) | 1 | (2.6) |
| | (%) | | | | | | | | | | |
| | Black or | 4 | (11.4) | 4 | (11.4) | 3 | (9.1) | 3 | (9.4) | 1 | (2.6) |
| | African | | | | | | | | | | |
| | American, n | | | | | | | | | | |
| | (%) | | | | | | | | | | |
| | White, n | 28 | (80.0) | 28 | (80.0) | 27 | (81.8) | 26 | (81.3) | 35 | (92.1) |
| | (%) | | | | | | | | | | |
| | Other, n | 1 | (2.9) | 1 | (2.9) | 1 | (3.0) | 1 | (3.1) | 1 | (2.6) |
| | (%) | | | | | | | | | | |
| Height (cm) | Mean (SD) | 177 | (6.5) | 177 | (6.5) | 177 | (6.6) | 177 | (6.7) | 179 | (6.7) |
| Weight (kg) | Mean (SD) | 84.3 | (11.5) | 84.3 | (11.5) | 83.2 | (10.8) | 83.5 | (10.9) | 83.3 | (13.0) |
| BMI (kg/m²) | Mean (SD) | 26.8 | (3.00) | 26.8 | (3.00) | 26.5 | (2.85) | 26.7 | (2.80) | 25.9 | (3.36) |

SD = standard deviation

### Drug-Drug Interactions

PK parameters when belumosudil was administered alone and in combination with itraconazole, rabeprazole, rifampicin, and omeprazole are displayed in Tables 3A-3C for the analytes belumosudil (Table 3A), KD025m2 (Table 3B), and KD025 ml (Table 3C). Results of statistical analyses conducted to evaluate drug interactions between belumosudil and each combination drug are presented in Table 4.

TABLES 3A-3C: PK Parameters of Belumosudil Following Administration With and Without CYP3A Compounds and PPIs

TABLE 3A
Analyte = Belumosudil

| | Belumosudil 200 mg QD (Part 1) | | | | Belumosudil 200 mg BID (Part 2) | |
|---|---|---|---|---|---|---|
| | Belumosudil | Belumosudil + | Belumosudil + | Belumosudil + | Belumosudil | Belumosudil + |
| | Alone | Itraconazole | Rifampicin | Rabeprazole | Alone | Omeprazole |
| Parameter | (N = 35) | (N = 35) | (N = 32) | (N = 32) | (N = 38) | (N = 38) |
| Cₘₐₓ | 1770 (31.3); | 2130 (31.0); | 712 (35.0) ^{c}; | 227 (58.4); | first dose: | first dose: |
| (ng/mL) | 1850 (572) | 2230 (684) | 753 (264) ^{c} | 262 (165) | 1790 (34.5) ^{k}; | 575 (145.7); |
| | | | | | 1880 (610) ^{k} | 893 (783) |
| | | | | | second dose: | second dose: |
| | | | | | 1760 (37.0); | 903 (63.8) ^{k}; |
| | | | | | 1870 (753) | 1050 (568) ^{k} |
| AUCₗₐₛₜ | 8750 (32.9); | 10900 (37.5); | 2440 (34.7) ^{c}; | 1580 (67.7); | 18900 (39.3) ^{k}; | 9680 (57.3) ^{k}; |
| (ng*h/mL) | 9220 (3240) | 11600 (4150) | 2580 (912) ^{c} | 1850 (973) | 20300 (8500) ^{k} | 10900 (5110) ^{k} |
| AUC_{inf} | 9080 (33.3) ^{a}; | 11200 (38.1) ^{b}; | 2500 (34.5) ^{c}; | 1680 (65.8) ^{d}; | 18800 (37.1) ^{a}; | 9880 (59.2) ^{m}; |
| (ng*h/mL) | 9580 (3400) ^{a} | 11900 (4310) ^{b} | 2640 (936) ^{c} | 1940 (978) ^{d} | 20100 (7630) ^{a} | 11200 (5360) ^{m} |
| AUC₀₋₂₄ | 8430 (30.5); | 10400 (35.8); | 2490 (34.1) ^{c}; | 1510 (60.7); | 16800 (36.4) ^{k}; | 7970 (58.9) ^{k}; |
| (ng*h/mL) | 8830 (2900) | 11000 (3770) | 2630 (916) ^{c} | 1730 (871) | 17900 (6980) ^{k} | 9100 (4560) ^{k} |
| Tₘₐₓ (h) | 3.00 (1.50-5.00) | 3.00 (1.50-5.00) | 3.00 (1.50-5.00) | 5.00 (2.00-6.00) | first dose: | first dose: |
| | | | | | 3.00 (1.00-6.00) | 4.00 (1.00-6.00) |
| | | | | | second dose: | second dose: |
| | | | | | 15.00 (13.00-20.00) | 17.00 (13.50-22.30) |
| t_{1/2} (h) | 7.89 (64.0) ^{a}; | 8.27 (36.3) ^{b}; | 2.17 (38.9) ^{c}; | 7.80 (77.3) ^{c}; | 6.86 (31.6) ^{l}; | 6.16 (37.6) ^{m}; |
| | 9.28 (5.63) ^{a} | 8.79 (3.20) ^{b} | 2.36 (1.27) ^{c} | 10.4 (12.3) ^{c} | 7.15 (1.96) ^{l} | 6.55 (2.26) ^{m} |
| CL/F | 367 (33.3) ^{a}; | 298 (38.1) ^{b}; | 1330 (34.5) ^{c}; | 1980 (65.8) ^{d}; | NR | NR |
| (mL/min) | 385 (116) ^{a} | 318 (117) ^{b} | 1400 (450) ^{c} | 2480 (2410) ^{d} | | |

TABLE 3B
Analyte = KD025m2

| | Belumosudil 200 mg QD (Part 1) | | | | Belumosudil 200 mg BID (Part 2) | |
|---|---|---|---|---|---|---|
| | Belumosudil | Belumosudil + | Belumosudil + | Belumosudil + | Belumosudil | Belumosudil + |
| | Alone | Itraconazole | Rifampicin | Rabeprazole | Alone | Omeprazole |
| Parameter | (N = 35) | (N = 35) | (N = 32) | (N = 32) | (N = 38) | (N = 38) |
| Cₘₐₓ | 337 (54.7); | 221 (60.8); | 148 (57.3) ^{c}; | 23.5 (70.7) ^{c}; | first dose: | first dose: |
| (ng/mL) | 379 (182) | 257 (152) | 169 (85.8) ^{c} | 29.4 (23.8) ^{c} | 282 (75.6) ^{k}; | 72.3 (166.8) ^{l}; |
| | | | | | 343 (202) ^{k} | 129 (133) ^{l} |
| | | | | | second dose: | second dose: |
| | | | | | 257 (83.9); | 115 (121.8) ⁿ; |
| | | | | | 323 (207) | 172 (160) ⁿ |
| AUCₗₐₛₜ | 1050 (68.1); | 704 (75.5); | 349 (61.1) ^{c}; | 85.8 (81.6) ^{v}; | 2030 (91.1) ^{k}; | 783 (122.6) ^{k}; |
| (ng*h/mL) | 1260 (811) | 891 (710) | 407 (237) ^{c} | 108 (76.5) ^{v} | 2690 (2090) ^{k} | 1160 (998) ^{k} |
| AUC_{inf} | 1230 (69.5) ^{d}; | 843 (73.7) ^{c}; | 396 (50.2) ^{h}; | 174 (11.1) ^{t}; | 1900 (90.4) ^{c}; | 1110 (90.3) ^{q}; |
| (ng*h/Ml) | 1460 (910) ^{d} | 1050 (792) ^{c} | 444 (237) ^{h} | 175 (19.4) ^{t} | 2550 (2200) ^{c} | 1430 (981) ^{q} |
| AUC₀₋₂₄ | 1100 (63.7); | 745 (70.7); | 376 (56.7) ^{c}; | 110 (84.0) ^{v}; | 1950 (82.3) ^{k}; | 767 (115.3) ^{k}; |
| (ng*h/mL) | 1290 (760) | 916 (659) | 431 (238) ^{c} | 139 (96.7) ^{v} | 2480 (1770) ^{k} | 1090 (850) ^{k} |
| Tₘₐₓ (h) | 3.00 (1.50-5.00) | 3.00 (2.00-5.00) | 3.00 (1.50-5.00) | 4.00 (2.00-6.00) | first dose: | first dose: |
| | | | | | 3.00 (1.00-6.00) | 4.00 (1.00-6.00) |
| | | | | | second dose: | second dose: |
| | | | | | 15.00 (13.45-20.00) | 17.00 (14.00-22.30) |
| t_{1/2} (h) | 3.03 (76.8) ^{d} | 2.89 (92.2) ^{g} | 1.27 (26.8) ^{h} | 3.10 (69.6) ^{w} | 4.32 (78.9) ^{c} | 2.76 (55.1) ^{r} |
| | 3.86 (3.24) ^{d} | 4.00 (3.69) ^{g} | 1.31 (0.368) ^{h} | 3.59 (1.84) ^{w} | 5.70 (5.24) ^{c} | 3.20 (2.10) ^{r} |

TABLE 3C
Analyte = KD025m1

| | Belumosudil 200 mg QD (Part 1) | | | | Belumosudil 200 mg BID (Part 2) | |
|---|---|---|---|---|---|---|
| | Belumosudil | Belumosudil + | Belumosudil + | Belumosudil + | Belumosudil | Belumosudil + |
| | Alone | Itraconazole | Rifampicin | Rabeprazole | Alone | Omeprazole |
| Parameter | (N = 35) | (N = 35) | (N = 32) | (N = 32) | (N = 38) | (N = 38) |
| Cₘₐₓ | 23.2 (40.0) ^{b}; | NC | 52.5 (34.0) ^{c}; | NC | first dose: | first dose: |
| (ng/mL) | 24.9 (9.21) ^{b} | | 55.2 (17.1) ^{c} | | 23.8 (39.6) ⁿ; | 18.0 (54.6) ^{j}; |
| | | | | | 25.8 (12.6) ⁿ | 20.5 (11.4) ^{j} |
| | | | | | second dose: | second dose: |
| | | | | | 23.6 (46.4) ⁿ; | 18.9 (48.7) ^{s}; |
| | | | | | 26.1 (12.7) ⁿ | 21.2 (11.3) ^{s} |
| AUCₗₐₛₜ | 55.3 (36.4) ^{e}; | NC | 126 (41.7) ^{c}; | NC | 157 (93.0) ^{l}; | 123 (114.0) ^{q}; |
| (ng*h/mL) | 58.4 (18.7) ^{e} | | 136 (55.5) ^{c} | | 205 (153) ^{l} | 164 (100) ^{q} |
| AUC_{inf} | NC | NC | 187 (20.0) ⁱ; | NC | 259 (47.7) ^{o}; | 265 (47.6) ^{t}; |
| (ng*h/mL) | | | 190 (37.7) ⁱ | | 287 (149) ^{o} | 279 (122) ^{t} |
| AUC₀₋₂₄ | 75.6 (42.1) ^{e}; | NC | 148 (41.9) ^{c}; | NC | 138 (91.3) ^{l}; | 91.5 (76.3) ^{q}; |
| (ng*h/mL) | 81.3 (30.7) ^{e} | | 159 (61.2) ^{c} | | 187 (161) ^{l} | 113 (81.2) ^{q} |
| Tₘₐₓ (h) | 3.00 (1.00-5.00) | NC | 2.08 (1.50-5.00) | NC | first dose: | first dose: |
| | | | | | 2.00 (1.00-6.00) | 3.00 (1.00-5.00) |
| | | | | | second dose: | Second dose: |
| | | | | | 13.98 (13.00-24.00) | 16.50 (13.50-22.00) |
| t_{1/2} (h) | 1.82 (38.5) ^{f}; | NC | 1.51 (21.4) ^{j}; | NC | 2.19 (34.2) ^{p}; | 2.21 (39.0) ^{u}; |
| | 1.96 (0.931) ^{f} | | 1.55 (0.325) ^{j} | | 2.30 (0.757) ^{p} | 2.31 (0.848) ^{u} |

In Tables 3A-3C, all parameters besides T. are presented as geometric mean (geometric CV %); arithmetic mean (standard deviation). T_is presented as median (range).

N=number of subjects in the PK analysis dataset; n=number of subjects with an observation; NC=not calculated; NR=not reported

^{a}n=34; ^{b}n=33; ^{c}n=29; ^{d}n=28; ^{e}n=27;^{f}n=11; ^{g}n=31; ^{h}n=23;ⁱn=13; ^{j}n=19; ^{k}n=37; ^{l}n=35; ^{m}n=32; ⁿn=36; ^{o}n=12; ^{p}n=14; ^{q}n=15; ^{r}n=17; ^{s}n=20; ^{t}n=2; ^{u}n=3; ^{v}n=21; ^{w}n=7

TABLE 4
Statistical Analysis of Belumosudil Pk Parameters Following Administration of
Belumosudil Alone and With Itraconazole, Rifampicin, Rabeprazole, or Omeprazole

| | | GMR [90% CI] | | | |
|---|---|---|---|---|---|
| | | Belumosudil + | Belumosudil + | Belumosudil + | Belumosudil + |
| | | Itraconazole | Rifampicin | Rabeprazole | Omeprazole |
| Analyte | Parameter | (N = 35) | (N = 29) | (N = 32) | (N = 36) |
| Belumosudil | Cₘₐₓ | 1.20 [1.11, | 0.41 [0.37, | 0.13 [0.11, | first dose: |
| | (ng/mL) | 1.31] | 0.44] | 0.15] | 0.32 [0.24, |
| | | n = 35 | n = 29 | n = 32 | 0.43] |
| | | | | | second dose: |
| | | | | | 0.51 [0.42, |
| | | | | | 0.61] |
| | | | | | n = 36 |
| | AUC₀₋₂₄ | N/A | N/A | N/A | 0.47 [0.40, |
| | (ng*h/mL) | | | | 0.55] |
| | | | | | n = 36 |
| | AUCₗₐₛₜ | 1.25 [1.17, | 0.28 [0.26, | 0.18 [0.16, | 0.51 [0.44, |
| | (ng*h/mL) | 1.33] | 0.30] | 0.21] | 0.59] |
| | | n = 35 | n = 29 | n = 32 | n = 36 |
| | AUC_{inf} | 1.25 [1.17, | 0.28 [0.26, | 0.20 [0.18, | 0.54 [0.46, |
| | (ng*h/mL) | 1.33] | 0.30] | 0.23] | 0.62] |
| | | n = 32 | n = 28 | n = 27 | n = 29 |
| KD025m2 | Cₘₐₓ | 0.66 [0.58, | 0.45 [0.39, | 0.07 [0.06, | first dose: 0.27 |
| | (ng/mL) | 0.73] | 0.51] | 0.08] | [0.19, 0.38] |
| | | n = 35 | n = 29 | n = 29 | n = 33 |
| | | | | | second dose: |
| | | | | | 0.43 [0.33, |
| | | | | | 0.55] |
| | | | | | n = 35 |
| | AUCₗₐₛₜ | 0.67 [0.62, | 0.33 [0.30, | 0.06 [0.05, | 0.36 [0.29, |
| | (ng*h/mL) | 0.73] | 0.37] | 0.08] | 0.46] |
| | | n = 35 | n = 29 | n = 21 | n = 36 |
| | AUC_{inf} | 0.67 [0.60, | 0.32 [0.28, | NC | NC |
| | (ng*h/mL) | 0.75] | 0.36] | | |
| | | n = 22 | n = 19 | | |
| KD025m1 | Cₘₐₓ | NC | 2.29 [2.03, | NC | first dose: 0.69 |
| | (ng/mL) | | 2.59] | | [0.55, 0.86] |
| | | | n = 28 | | n = 19 |
| | | | | | second dose: |
| | | | | | 0.76 [0.62, |
| | | | | | 0.94] |
| | | | | | n = 20 |
| | AUCₗₐₛₜ | NC | 2.50 [2.23, | NC | NC |
| | (ng*h/mL) | | 2.81] | | |
| | | | n = 22 | | |
| | AUC_{inf} | NC | NC | NC | NC |
| | (ng*h/mL) | | | | |

CI = confidence interval;
GMR = adjusted geometric mean ratio;
N = number of subjects in the PK analysis dataset;
n = number of subjects with an observation;
N/A = not applicable;
NC = not calculated
Test: single dose 200 mg belumosudil QD + 200 mg itraconazole QD × 9 days, 20 mg rabeprazole QD × 4 days, or 600 mg rifampicin QD × 9 days (Part 1); 200 mg belumosudil BID on Day 1 + 20 mg omeprazole QD × 3 days (Part 2)
Reference: single dose 200 mg belumosudil QD administered alone (Part 1) or 200 mg belumosudil BID administered alone (Part 2)

### Effect of Itraconazole

Following administration of 200 mg belumosudil with and without 200 mg itraconazole (QD x 9 days), belumosudil was rapidly absorbed. Median Tₘₐₓ occurred 3 hours post-dose both with and without itraconazole (FIG. 2A). Co-administration of itraconazole with belumosudil resulted in geometric mean Cₘₐₓ, AUCₗₐₛₜ and AUC_{inf} values 1.2x those for belumosudil alone, with 90% CIs of the GMRs within the relative bioavailability limits of 70.00 to 143.00% (Table 4). Concomitant administration of itraconazole did not appear to reduce or elongate the elimination half-life of belumosudil. Concentrations of metabolite KD025 ml decreased to nonquantifiable levels in all but 1 subject following co-administration of belumosudil with itraconazole. Similarly, KD025m2 exposure decreased by 35%, 33%, and 33% for Cₘₐₓ, AUCₗₐₛₜ and AUC_{inf}, respectively (Tables 3A-3C).

### Effect of Rifampicin

Administration of 200 mg belumosudil with and without 600 mg rifampicin (QD x 9 days) resulted in rapid absorption of belumosudil, with median Tₘₐₓ observed 3 hours post-dose (FIG. 2A). Peak and overall belumosudil exposure levels decreased in the presence of rifampicin, as Cₘₐₓ, AUCₗₐₛₜ and AUC_{inf} were approximately 59%, 72%, and 72% lower, respectively, than those for belumosudil dosed alone. Concomitant administration of rifampicin also reduced the elimination half-life of belumosudil from a geometric mean of 7.89 hours without rifampicin to 2.17 hours with rifampicin. Compared to belumosudil alone, rifampicin increased exposure of metabolite KDO25 ml (Cₘₐₓ and AUCₗₐₛₜ) by 129% and 150%, respectively, and decreased exposure of metabolite KD025m2 by 55 to 67%.

### Effect of PPIs Rabeprazole and Omeprazole

Following concomitant administration of a single belumosudil 200 mg dose and 20 mg rabeprazole, a PPI, median Tₘₐₓ was delayed by 2 hours (FIGS. 2A and 2B) and mean belumosudil exposure was approximately 87% (Cₘₐₓ), 82% (AUCₗₐₛₜ), and 80% (AUC_{inf}) lower than when belumosudil was administered alone (Table 3A). For all parameters, 90% CIs did not fall within the limits of relative bioavailability in this study. Rabeprazole did not appear to have an effect on the elimination half-life of belumosudil. Like belumosudil, exposure of metabolites KD025 ml and KD025m2 decreased following co-administration of belumosudil with rabeprazole. KDO25 ml levels were nonquantifiable in all but 1 subject, and KD025m2 Cₘₐₓ and AUCₗₐₛₜ were decreased by 93% and 94%, respectively, in the presence of rabeprazole.

Similarly, treatment with 200 mg belumosudil BID and 20 mg omeprazole, a slightly weaker PPI than rabeprazole, resulted in a 1-to-2-hour delay in belumosudil absorption. In the presence of omeprazole, Cₘₐₓ for the first dose of belumosudil was 68% lower than for belumosudil alone; AUC0-24 was 53% lower. For all primary endpoints, 90% CIs did not fall within the relative bioavailability acceptance limits of 70.00 to 143.00%. Following administration of belumosudil with omeprazole, concentrations of KD025 ml were sparse and sporadic, with several subjects having nonquantifiable profiles following both dosing occasions. Concentrations of KD025m2 also decreased when belumosudil was concomitantly administered with omeprazole, with approximate decreases of 73%, 57%, and 63% for C_{max,first dose}, C_{max, second dose}, and AUC0-24, respectively.

### Safety

There were no deaths or AEs leading to subject withdrawal in the study. A total of 33 treatment-emergent AEs (TEAEs) were reported in Periods 1, 2, and 4, the majority of which were mild in severity (Table 3). Three AEs, all mild in severity, were considered possibly related to belumosudil: abdominal distension, hot flush, and fatigue. One serious AE (SAE) of ankle fracture was reported 11 days after administration of belumosudil+itraconazole. This SAE was severe and was considered unrelated to belumosudil. Gastrointestinal disorders were the most commonly reported System Organ Class, reported by 3 subjects following dosing with belumosudil alone and 5 subjects after dosing with belumosudil+itraconazole. Except for 1 moderate AE of neutropenia, which is a common occurrence in healthy subjects of African origin, there were no clinically significant laboratory findings.

### Analysis

This was a single center, non-randomized DDI study in healthy male subjects. The PK objectives of this study were to determine the effects of CYP3A4 inhibitors and inducers (itraconazole and rifampicin, respectively) and PPIs (rabeprazole and omeprazole) on the PK of belumosudil and its metabolites following oral administration in healthy male subjects. Itraconazole, a CYP3A4 index inhibitor, and rifampicin, a CYP3A4 index inducer, were selected as perpetrator compounds. Rabeprazole and omeprazole PPIs were selected to evaluate pH-dependent changes in belumosudil PK based on rabeprazole's ability to increase pH levels and omeprazole's widespread use in treatment of gastroesophageal reflux disease (GERD) and management of gastrointestinal involvement in patients with cGVHD.

Although itraconazole is classified as a strong CYP3A inhibitor, it does not induce maximal CYP3A inhibition compared with ketoconazole. However, at a dose regimen of 200 mg QD for 4 days, itraconazole has been demonstrated in the literature to achieve clinically relevant inhibition of CYP3A4. Kantola T, et al., Effect of itraconazole on the pharmacokinetics of atorvastatin. Clin Pharmacol Ther. 1998;64 (1): 58-65; Lebrun-Vignes B, et al., Effect of itraconazole on the pharmacokinetics of prednisolone and methylprednisolone and cortisol secretion in healthy subjects. Br J Clin Pharmacol. 2001;51 (5): 443-450.

In this study, a regimen of 200 mg QD for 9 days was utilized (from Day-7 through 1 day after belumosudil dosing [Day 2]). This 9-day duration was considered short enough to avoid the time-dependent half-life increases that occur after approximately 15 days of itraconazole administration. Hardin TC et al., Pharmacokinetics of itraconazole following oral administration to normal volunteers. Antimicrob Agents Chemother. 1988;32 (9): 1310-1313.

The dose level of 200 mg is further supported as it is the clinically recommended dose for the treatment of various fungal infections, as per the itraconazole prescribing information. While no meaningful change in belumosudil exposure was noted for administration with itraconazole (a CYP3A4 inhibitor), a decrease in exposure of KD025m2 was observed. Additionally, KD025 ml concentrations were reduced to mostly nonquantifiable levels. This supports preclinical data suggesting that CYP3A4 is likely to play a role in the metabolism of belumosudil to the ROCK2-active minor metabolite KD025 ml and the ROCK2-inactive major metabolite KD025m2.

Regarding rifampicin, a dose regimen of 600 mg QD for 5 to 9 days has been modeled by others and shown to produce steady-state induction of CYP3A4 activity. Because this is also the labeled dose regimen, 9 days of dosing prior to belumosudil administration was considered appropriate for this drug interaction study. Additionally, because rifampicin is also an organic ion transporter (OAT) inhibitor, belumosudil and rifampicin were not administered concomitantly on Day 1 in order to avoid confounding effects in the event that belumosudil was later identified as an OAT substrate. Co-administration with rifampicin (a CYP3A4 inducer) resulted in a significant decrease in belumosudil exposure (Cₘₐₓ, AUC_{inf}, and AUCₗₐₛₜ decreased by 59%, 72%, and 72%, respectively). In combination with an increase in KD025 ml exposure, these results indicate a higher extent of belumosudil metabolism with an induction of CYP3A4 activity. Geometric mean half-life values decreased from 7.89 (belumosudil alone) to 2.17 hours (with rifampicin); however, the decrease in half-life may be attributed to characterization of the distribution phase rather than true elimination phase of belumosudil, as belumosudil concentrations reached nonquantifiable levels earlier in the presence of rifampicin.

The effect of CYP3A4 induction on belumosudil metabolism is further supported by in vitro assessments, which determined that the metabolism of belumosudil to KD025 ml was CYP3A4-and CYP2C8-dependent, while metabolism to KD025m2 was CYP3A4-dependent, with further metabolism modulated by UGTIA1. As rifampicin is known to induce CYP3A4, CYP2C8, and UGTIAI (Chen J, et al., Roles of rifampicin in drug-drug interactions: underlying molecular mechanisms involving the nuclear pregnane X receptor. Ann Clin Microbiol Antimicrob. 2006; 5:3), the observed increase in KD025 ml exposure with rifampicin co-administration is consistent with the metabolic pathway of belumosudil. Additionally, whereas production of KDO25 ml may increase from induction of both CYP3A4 and CYP2C8, induction of the UGTIAl enzyme may increase the elimination rate of KD025m2 even if production via CYP3A4 is increased, thus explaining the decrease in exposure of KD025m2 with rifampicin administration.

Rabeprazole, a strong PPI, suppresses the secretion of gastric acid by noncompetitive blockade of H+/K+-adenosine triphosphatase at the secretory surface of gastric parietal cells, thereby raising the intra-gastric pH above 3.0. The recommended starting dose of sodium rabeprazole for subjects with GI ulcers and GERD is 20 mg per day and for subjects with hypersecretory syndromes is 60 mg per day (Accord-UK Ltd. Summary of Product Characteristics: Rabeprazole 20 mg Gastro-resistant Tablets, available at www.medicines.org.uk/emc/medicine/27143/SPC/Rabeprazole+20 mg+Gastro-resistant+Tablets/. Updated 16 Apr. 2020. Accessed 12 Aug. 2021). A dose regimen of 20 mg BID for 3 days followed by a QD dose prior to belumosudil administration was selected for this short-duration study. Omeprazole doses were administered at the lowest labeled dose (20 mg) in Part 2 of the study to investigate a weaker PPI interaction with belumosudil. Decreased exposures of belumosudil and its metabolites following co-administration with rabeprazole, a strong PPI, suggest that an increase in gastric pH resulted in decreased solubility of belumosudil, in turn reducing belumosudil absorption. Upon co-administration with a weaker PPI, omeprazole, similar trends in belumosudil and metabolite PK were observed. These findings are consistent with previously conducted solubility studies, showing a decrease in belumosudil solubility from >100 μg/mL at pH 2.7 to approximately 4 ug/mL at pH 6.5 and approximately 3 ug/mL at pH 7.4. Based on the results in this study, a clinically meaningful interaction between belumosudil and PPIs is possible; as such, the prescribing information dictates that belumosudil dosing should be increased when co-administered with PPIs.

It is noted that while omeprazole has the potential to induce CYP3A4, it is a relatively weak CYP3A4 inducer and was only administered in this study for a total of 4 days. As such, any DDIs are not considered to be due to CYP induction and instead are likely due to the pH-limited solubility of belumosudil.

The safety profile of belumosudil in this study was unremarkable for single oral doses of 200 mg belumosudil alone and in combination with 200 mg itraconazole or 600 mg rifampicin. The majority of TEAEs were mild in severity and all resolved by the end of the study. One SAE was reported; this event was unrelated to belumosudil.

Potential drug-drug interactions between belumosudil and itraconazole (a CYP3A4 inhibitor) and rifampicin (a CYP3A4 inducer) were explored. In the presence of itraconazole, no clinically meaningful changes in belumosudil PK were observed; however, concentrations of both belumosudil metabolites decreased relative to when belumosudil was administered alone. Rifampicin was found to have a significant effect on belumosudil PK, as observed by a decrease in belumosudil and KD025m2 exposure and an increase in KD025 ml exposure with rifampicin. Co-administration of both the PPI rabeprazole and a weaker PPI, omeprazole, with belumosudil resulted in significant decreases in belumosudil and metabolite exposure; as such, belumosudil is recommended for dosing at a higher level when co-administered with PPIs. No safety or tolerability risks were identified for single doses of belumosudil administered alone and in combination with repeat doses of itraconazole, rifampicin, omeprazole, or rabeprazole.

### Example 2: Two Phase 1 Studies to Evaluate the Food Effect and Relative Bioavailability of Tablet and Capsule Formulations of Belumosudil in Healthy Adult Subjects

The interaction between food and the pharmacokinetic profile of an orally administered medication is influenced by many factors. The mechanisms that drive pharmacokinetic food-drug interactions largely depend on the drug substance and formulation, impact on gastrointestinal physiology (e.g., pH, milieu, emptying rate), and meal composition (caloric and fat content). Food effect represents a complex challenge in oral drug administration.

To evaluate the pharmacokinetic (PK) differences between the 2 formulations and determine the presence of a potential food effect of belumosudil, two Phase 1 studies were conducted. The first study was an initial preliminary food effect study assessing administration of belumosudil capsules with food. The following study was a relative bioavailability and food effect study to compare the marketed tablet and capsule formulations, in addition to characterizing the food effect for belumosudil tablets. This Example reports the PK and safety results from both studies.

### Study Design

Both studies were single-center, open-label, randomized, single dose crossover studies in healthy males. The key inclusion criteria included subjects aged between 18 and 55 years (inclusive) and who were using a highly effective method of birth control both during the study and for 1 to 3 months after the study. For both studies, the API was belumosudil mesylate, equivalent to 100 mg (capsules) or 200 mg (tablets) free base.

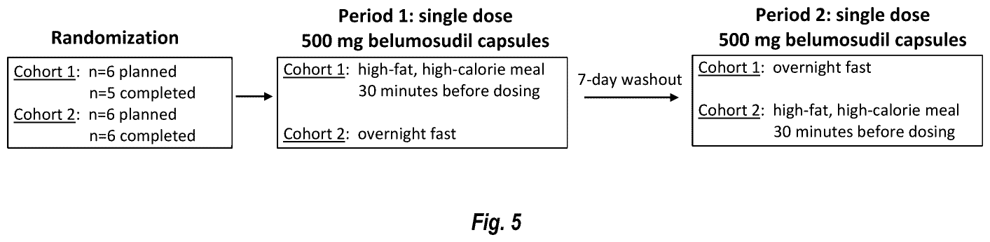

In the first food effect study, a dose of 500 mg belumosudil (5×100 mg capsules) was selected due to the absence of drug-related toxicities at that dose in a previous single and multiple ascending dose (SAD/MAD) study. Twelve healthy males were enrolled in the study and were randomized to receive a single dose of 5×100 mg belumosudil capsules under both fed and fasted conditions in a crossover fashion. This study design is shown in FIG. 5 herein.

For the fed condition, subjects were given a high-fat meal consisting of fried eggs, bacon, buttered toast, hash browns, and 8 ounces of whole milk 30 minutes prior to receiving belumosudil. For the fasted condition, subjects were fasted overnight for ≥10 hours prior to receiving belumosudil and for 4 additional hours post-dose. Water was permitted ad libitum except for 1 hour pre-and post-dose. A 7-day washout period separated each dose of belumosudil that was administered under either fed or fasted conditions.

In the second, relative bioavailability/food effect study, both the marketed tablets and earlier formulation capsules were investigated. A 200 mg tablet dose was selected as it was well tolerated in healthy subjects and produced quantifiable concentrations across the full PK profile. Additionally, because belumosudil tablets and capsules were manufactured in 200 mg and 100 mg dose units, respectively, the 200 mg dose allowed for precise comparative variability assessments between the two formulations. A total of 23 healthy males were enrolled in the study and were randomized to receive 3 single doses of 200 mg belumosudil according to their assigned treatment sequence. These demographics are shown in Table 5.

TABLE 5
Demographics of Clinical Study, Example 2 (Second Study)

| | | | Relative BA/ | |
|---|---|---|---|---|
| | Food Effect Study | | Food Effect Study | |
| | (Capsule) | | (Tablet and Capsule) | |
| | KD025-105 | | KD025-106 | |
| Population | N = 12 | | N = 23 | |
| Safety Population | 12 | (100.0%) | 23 | (100.0%) |
| PK Population | 12 | (100.0%) | 23 | (100.0%) |
| Completed study | 11 | (91.7%) | 19 | (82.6%) |
| Discontinued prematurely: | 1 | (8.3%) | 4 | (17.4%) |
| Serious or Severe Adverse Event | 0 | (0.0%) | 0 | (0.0%) |
| Other AE: Significant Liver Elevation | 0 | (0.0%) | 2 | (8.7%) |
| Protocol Deviation | 0 | (0.0%) | 0 | (0.0%) |
| Lost to Follow-up | 1 | (8.3%) | 0 | (0.0%) |
| Withdrawal by Subject | 0 | (0.0%) | 1 | (4.3%) |
| Other: Illness of prohibited medication | 0 | (0.0%) | 1 | (4.3%) |

| Demographics | Mean (SD) or n (%) | | | |
|---|---|---|---|---|
| Age (years) | 31.8 | (11.8) | 36.2 | (11.5) |
| Weight (kg) | 80.3 | (11.8) | 81.2 | (10.0) |
| BMI (kg/m²) | 25.6 | (2.7) | 26.2 | (2.7) |
| Race: White | 4 | (33.3%) | 20 | (87.0%) |
| Race: Black or | 8 | (66.7%) | 3 | (13.0%) |
| African American | | | | |

BA = bioavailability;
BMI = body mass index;
PK = pharmacokinetic;
SD = standard deviation

For Treatment A, subjects were administered a 200 mg belumosudil tablet after an overnight fast, then continued to fast for 4 additional hours post-dose. For Treatments B and C, before dosing, subjects consumed a high-fat breakfast consisting of a hash brown, bacon, fried egg, white bread, and 240 mL of full fat milk over 25 minutes. Thirty minutes after the start of breakfast, subjects received a single dose of either a 200 mg belumosudil tablet (Treatment B) or 2×100 mg belumosudil capsules (Treatment C). All subjects were to receive each treatment, with at least a 6-day washout between each period. Water was permitted ad libitum in all study periods, besides for 1 hour pre-and post-dose.

### Pharmacokinetic Sample Collection

In both studies, serial blood samples for PK analysis were collected at pre-dose and 1, 2, 4, 6, 8, 12, 16, 24, and 36 hours post-dose. An additional sample at 30 hours post-dose was collected in the first study; additional samples were collected 3, 5, and 48 hours post-dose in the second study. Blood samples (5-6 mL per sample) were collected in K3EDTA tubes on ice and were processed within 30 minutes of collection, then frozen at approximately-70 to −80° C. shortly after collection.

Sample preparation was performed by protein precipitation. Belumosudil and its 2 metabolites (KD025 ml and KD025m2) were quantified using a liquid chromatography tandem mass spectrometry (LC-MS/MS) method validated from 10.0 to 5000 ng/mL.

### Pharmacokinetic and Statistical Analysis Methods

The primary endpoint for the first, initial preliminary food effect study was safety and tolerability, with a secondary endpoint of PK for belumosudil and its metabolites (KD025 ml and KD025m2). For the second, relative bioavailability/food effect study, the primary endpoint was a comparison of PK parameters for belumosudil administered as tablet in the fed versus fasted state. Secondary endpoints included PK comparison of belumosudil administered as a tablet versus capsule formulation, as well as safety and tolerability assessments.

Statistical analyses were performed in SAS using the PROC MIXED procedure and the Restricted Maximum Likelihood estimation method. In both studies, the presence of a food effect was evaluated by a formal statistical analysis on natural log-transformed AUC and Cₘₐₓ using mixed effect modeling techniques. The model included treatment, period, and sequence as fixed effects and subject nested within sequence as a random effect. Adjusted geometric mean ratios (GMRs) and their 90% confidence intervals (CIs) were calculated for fed (test) versus fasted (reference) treatments. The absence of a food effect was to be concluded if the 90% CI was within the bioequivalence limits of 80.00 to 125.00%.

To assess relative bioavailability between formulations in the second study, the same analysis was performed for tablets (test) versus capsules (reference), both in the fed state. For both studies, all subjects who received at least one dose of study drug and had post-dose PK data available were included in the PK population

### Results

Pharmacokinetic Results

In the initial, preliminary food effect study, after administration of a single oral dose of belumosudil 500 mg (capsules) both fed and fasted, belumosudil was rapidly absorbed with no lag time observed. Cₘₐₓ was achieved 2 hours earlier under fasted conditions as compared to fed. After reaching peak concentrations, mean plasma concentrations of belumosudil declined in a similar manner between the two treatments.

Both Cₘₐₓ and AUC of belumosudil were higher in the fed state than in the fasted state, with GMRs above 125% for all PK parameters included in the statistical analysis. Between subject variability in Cₘₐₓ and AUC was slightly lower for the fasted state than the fed state. Similar trends were observed for metabolite data.

In the second, relative bioavailability/food effect study, administration of the 200 mg tablet with a high-fat breakfast reduced variability and increased belumosudil exposure to 225% (Cₘₐₓ) and 180% (AUC_{inf}) of that observed under fasted conditions. A 0.5 h delay in Tₘₐₓ was observed with fed administration. Statistical analysis of the GMRs further supported the presence of a food effect. Circulating metabolite levels were low regardless of formulation and fed status.

These results from both the first and second studies are summarized below in Table 6, which shows the pharmacokinetic parameters for Belumosudil in the two food effect studies, and the relative bioavailability data in the second study.

TABLE 6
Pharmacokinetic Parameters for Belumosudil in Two Food Effect Studies

| | 5 × 100 mg Capsules | | 200 mg Tablet | |
|---|---|---|---|---|
| | (Study 1) | | (Study 2) | |
| Parameter | Fed, N = 12 | Fasted, N = 12 | Fed, N = 20 | Fasted, N = 23 |
| AUCₗₐₛₜ (ng*h/mL) | 24400 (42.8); | 8190 (31.6); | 9750 (49.3); | 4520 (121.3); |
| | 26250 (9770) | 8586 (3111) | 10800 (4970) | 5830 (3440) |
| AUC_{inf} (ng*h/mL) | 25106 (42.3); | 8312 (29.8)^{a}; | 10100 (52.9)^{b}; | 4910 (146.9)^{b}; |
| | 26925 (9902) | 8701 (3331)^{a} | 11300 (5340)^{b} | 6670 (4070)^{b} |
| AUC₀₋₂₄ (ng*h/mL) | 23311 (43.1); | 7603 (31.8); | 9580 (48.4); | 4250 (106.5); |
| | 25067 (9410) | 7976 (2848) | 10600 (4790) | 5400 (3210) |
| AUC_{% extrap} (%) | NR | NR | 1.55 (50.3); | 6.02 (90.8); |
| | | | 1.71 (0.71) | 8.38 (9.05) |
| Cₘₐₓ (ng/mL) | 3780 (59.3); | 1503 (34.3); | 2100 (49.8); | 821 (129.5); |
| | 4284 (2064) | 1580 (514.5) | 2320 (1040) | 1110 (734) |
| t_{1/2} (h) | 7.9 (23.9); | 9.0 (34.9) ^{a}; | 6.98 (44.5)^{b}; | 11.16 (48.6)^{b}; |
| | 8.0 (1.83) | 9.5 (3.22)^{a} | 7.59 (3.29)^{b} | 12.16 (4.92)^{b} |
| Tₘₐₓ (h) | 4.0 (4.0-8.0) | 2.0 (1.0-4.0) | 2.50 (1.00-6.00) | 2.00 (1.00-5.00) |

All parameters besides Tₘₐₓ are presented as geometric mean (geometric CV %); arithmetic mean (SD). Tₘₐₓ is presented as median (range).
NR = not reported
^{a}N = 10 for AUC_{inf} and N = 9 for t_{1/2} in fasted condition
^{b}N = 14 for fed condition; N = 17 for fasted condition

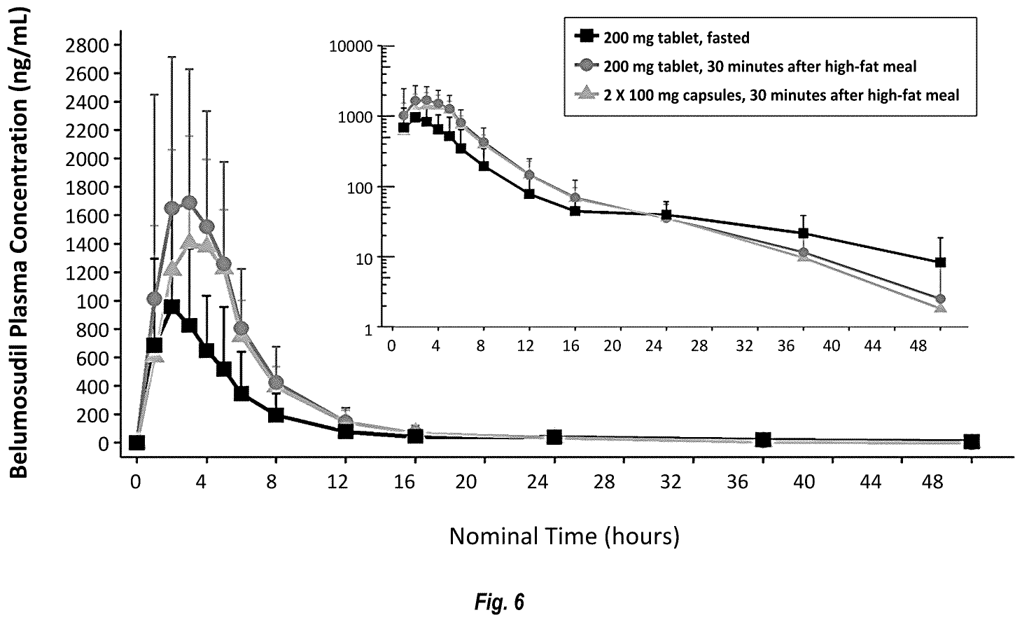

As illustrated in FIG. 6 herein, following a single dose of 200 mg belumosudil in the fed state, belumosudil exposure was slightly higher for the tablet versus capsule. Variability in exposure between subjects was moderate for both the tablet and capsule but was slightly higher for the tablet. Belumosudil exposure (Cₘₐₓ and AUC) was approximately 17-19% higher for the tablet versus capsule.

These results demonstrate that fed administration decreases the rate and increases the extent of belumosudil absorption. This was first evidenced in the initial food effect study, where administration of a high-fat meal 30 minutes before belumosudil dosing significantly increased systemic exposure (Cₘₐₓ, AUCO-t, and AUC_{inf}) to 2.5x-3x that of belumosudil when administered in the fasted state. Tₘₐₓ was achieved 2 hours later under fed conditions.

Comparable findings of increased rate and extent of absorption were noted in the second food effect study, where administration of the 200 mg tablet under fed conditions delayed Tₘₐₓ by 0.5 hours and increased belumosudil exposure to approximately 2x that of tablet exposure under fasted conditions. Moreover, inter-subject variability in exposure was reduced by approximately 60% when the tablet was administered with food. These results demonstrate that the belumosudil tablet formulation slightly minimizes food effects on PK as compared to the capsule formulation and that food improves consistency of exposure across subjects.

In addition to food effect, the relative bioavailability of the two formulations was assessed under fed conditions. For both the tablet and capsule, belumosudil was quickly absorbed and eliminated, as evidenced by rapid appearance of metabolites KD025 ml and KD025m2. Variability, as assessed by CV % of exposure parameters, was similar, suggesting no marked differences in inter-subject variability in PK between the two formulations.

### Example 3: United States REZUROCK™ (belumosudil) FDA Label

### INDICATIONS AND USAGE

REZUROCK is a kinase inhibitor indicated for the treatment of adult and pediatric patients 12 years and older with chronic graft-versus-host disease (chronic GVHD) after failure of at least two prior lines of systemic therapy. (1)

### DOSAGE AND ADMINISTRATION

Recommended Dosage: 200 mg taken orally once daily with food. (2.1)

### DOSAGE FORMS AND STRENGTHS

Tablet: 200 mg. (3)

### CONTRAINDICATIONS

### None. (4)

### WARNINGS AND PRECAUTIONS

Embryo-Fetal Toxicity: Can cause fetal harm. Advise females of reproductive potential of the potential risk to a fetus and to use effective contraception. (5.1, 8.1, 8.3)

### DRUG INTERACTIONS

Strong CYP3A Inducers: Increase REZUROCK dosage to 200 mg twice daily. (7.1) Proton Pump Inhibitors: Increase REZUROCK dosage to 200 mg twice daily. (7.1)

### ADVERSE REACTIONS

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension. (6.1)

### USE IN SPECIFIC POPULATIONS

Lactation: Advise not to breastfeed. (8.2) See 17 for PATIENT COUNSELING INFORMATION and FDA-approved patient labeling.

### FULL PRESCRIBING INFORMATION

1 INDICATIONS AND USAGE

REZUROCK is indicated for the treatment of adult and pediatric patients 12 years and older with chronic graft-versus-host disease (chronic GVHD) after failure of at least two prior lines of systemic therapy.

2 DOSAGE AND ADMINISTRATION

2.1 Recommended Dosage

The recommended dose of REZUROCK is 200 mg given orally once daily until progression of chronic GVHD that requires new systemic therapy.

Instruct the patient on the following:

Swallow REZUROCK tablets whole. Do not cut, crush, or chew tablets.

Take REZUROCK with a meal at approximately the same time each day [see Clinical Pharmacology (12.3)].

If a dose of REZUROCK is missed, instruct the patient to not take extra doses to make up the missed dose.

Treatment with REZUROCK has not been studied in patients with pre-existing severe renal or hepatic impairment. For patients with pre-existing severe renal or hepatic impairment, consider the risks and potential benefits before initiating treatment with REZUROCK [see Clinical Pharmacology (12.3)].

2.2 Dose Modifications for Adverse Reactions

Monitor total bilirubin, aspartate aminotransferase (AST), and alanine aminotransferase (ALT) at least monthly. Modify the REZUROCK dosage for adverse reactions as per Table 7.

TABLE 7
Recommended Dosage Modifications for REZUROCK for Adverse Reactions

| Adverse Reaction | Severity* | REZUROCK Dosage Modifications |
|---|---|---|
| Hepatotoxicity [see | Grade 3 AST or ALT | Hold REZUROCK until recovery of |
| Adverse Reactions | (5x to 20x ULN) or | bilirubin, AST and ALT to Grade 0-1, |
| (6.1)] | Grade 2 bilirubin | then resume REZUROCK at the |
| | (1.5x to 3x ULN) | recommended dose. |
| | Grade 4 AST or ALT | Discontinue REZUROCK permanently. |
| | (more than 20x ULN) or | |
| | Grade ≥ 3 bilirubin | |
| | (more than 3x ULN) | |
| Other adverse reactions | Grade 3 | Hold REZUROCK until recovery to |
| [see Adverse Reactions | | Grade 0-1, then resume REZUROCK at |
| (6.1)] | | the recommended dose level. |
| | Grade 4 | Discontinue REZUROCK permanently. |

*Based on CTCAE v 4.03

2.3 Dosage Modification Due to Drug Interactions

### Strong CYP3A Inducers

Increase the dosage of REZUROCK to 200 mg twice daily when coadministered with strong CYP3A inducers [see Drug Interactions (7.1)].

### Proton Pump Inhibitors

Increase the dosage of REZUROCK to 200 mg twice daily when coadministered with proton pump inhibitors [see Drug Interactions (7.1)].

3 DOSAGE FORMS AND STRENGTHS

Each 200 mg tablet is a pale yellow film-coated oblong tablet debossed with “KDM” on one side and “200” on the other side.

4 CONTRAINDICATIONS

None.

5 WARNINGS AND PRECAUTIONS

5.1 Embryo-Fetal Toxicity

Based on findings in animals and its mechanism of action, REZUROCK can cause fetal harm when administered to a pregnant woman. In animal reproduction studies, administration of belumosudil to pregnant rats and rabbits during the period organogenesis caused adverse developmental outcomes including embryo-fetal mortality and malformations at maternal exposures (AUC) less than those in patients at the recommended dose. Advise pregnant women of the potential risk to a fetus. Advise females of reproductive potential and males with female partners of reproductive potential to use effective contraception during treatment with REZUROCK and for at least one week after the last dose [see Use in Specific Populations (8.1, 8.3), Nonclinical Toxicology (13.1)].

6 ADVERSE REACTIONS

6.1 Clinical Trial Experience

Because clinical trials are conducted under widely variable conditions, adverse reaction rates observed in clinical trials of a drug cannot be directly compared with rates of clinical trials of another drug and may not reflect the rates observed in practice.

Chronic Graft versus Host Disease

In two clinical trials (Study KD025-213 and Study KD025-208), 83 adult patients with chronic GVHD were treated with REZUROCK 200 mg once daily [see Clinical Studies (14.1)]. The median duration of treatment was 9.2 months (range 0.5 to 44.7 months).

Fatal adverse reaction was reported in one patient with severe nausea, vomiting, diarrhea and multi-organ failure.

Permanent discontinuation of REZUROCK due to adverse reactions occurred in 18% of patients. The adverse reactions which resulted in permanent discontinuation of REZUROCK in >3% of patients included nausea (4%). Adverse reactions leading to dose interruption occurred in 29% of patients. The adverse reactions leading to dose interruption in ≥2% were infections (11%), diarrhea (4%), and asthenia, dyspnea, hemorrhage, hypotension, liver function test abnormal, nausea, pyrexia, edema, and renal failure with (2% each).

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension.

Table 8 summarizes the nonlaboratory adverse reactions.

TABLE 8
Nonlaboratory Adverse Reactions in ≥10% Patients
with Chronic GVHD Treated with REZUROCK

| | REZUROCK | |
|---|---|---|
| | 200 mg once daily (N = 83) | |
| Adverse Reaction | All Grades (%) | Grades 3-4 (%) |

Infections and infestations

| | | |
|---|---|---|
| Infection (pathogen not specified)^{a} | 53 | 16 |
| Viral infection^{b} | 19 | 4 |
| Bacterial infection^{c} | 16 | 4 |

General disorders and administration site conditions

| | | |
|---|---|---|
| Asthenia^{d} | 46 | 4 |
| Edema^{e} | 27 | 1 |
| Pyrexia | 18 | 1 |

Gastrointestinal

| | | |
|---|---|---|
| Nausea^{f} | 42 | 4 |
| Diarrhea | 35 | 5 |
| Abdominal pain^{g} | 22 | 1 |
| Dysphagia | 16 | 0 |

Respiratory, thoracic and mediastinal

| | | |
|---|---|---|
| Dyspnea^{h} | 33 | 5 |
| Coughⁱ | 30 | 0 |
| Nasal congestion | 12 | 0 |

Vascular

| | | |
|---|---|---|
| Hemorrhage^{j} | 23 | 5 |
| Hypertension | 21 | 7 |

Musculoskeletal and connective tissue

| | | |
|---|---|---|
| Musculoskeletal pain^{k} | 22 | 4 |
| Muscle spasm | 17 | 0 |
| Arthralgia | 15 | 2 |

Nervous system

| | | |
|---|---|---|
| Headache^{l} | 21 | 0 |

Metabolism and nutrition

| | | |
|---|---|---|
| Decreased appetite | 17 | 1 |

Skin and subcutaneous

| | | |
|---|---|---|
| Rash^{m} | 12 | 0 |
| Pruritusⁿ | 11 | 0 |

^{a}infection with an unspecified pathogen includes acute sinusitis, device related infection, ear infection, folliculitis, gastroenteritis, gastrointestinal infection, hordeolum, infectious colitis, lung infection, skin infection, tooth infection, urinary tract infection, wound infection, upper respiratory tract infection, pneumonia, conjunctivitis, sinusitis, respiratory tract infection, bronchitis, sepsis, septic shock.
^{b}includes influenza, rhinovirus infection, gastroenteritis viral, viral upper respiratory tract infection, bronchitis viral, Epstein-Barr viremia, Epstein-Barr virus infection, parainfluenzae virus infection, Varicella zoster virus infection, viral infection.
^{c}includes cellulitis, Helicobacter infection, Staphylococcal bacteremia, catheter site cellulitis, Clostridium difficile colitis, Escherichia urinary tract infection, gastroenteritis Escherichia coli, Pseudomonas infection, urinary tract infection bacterial.
^{d}includes fatigue, asthenia, malaise.
^{e}includes edema peripheral, generalized edema, face edema, localized edema, edema.
^{f}includes nausea, vomiting.
^{g}includes abdominal pain, abdominal pain upper, abdominal pain lower.
^{h}includes dyspnea, dyspnea exertional, apnea, orthopnea, sleep apnea syndrome.
ⁱincludes cough, productive cough.
^{j}includes contusion, hematoma, epistaxis, increased tendency to bruise, conjunctival hemorrhage, hematochezia, mouth hemorrhage, catheter site hemorrhage, hematuria, hemothorax, purpura.
^{k}includes pain in extremity, back pain, flank pain, limb discomfort, musculoskeletal chest pain, neck pain, musculoskeletal pain.
^{l}includes headache, migraine.
^{m}includes rash, rash maculo-papular, rash erythematous, rash generalized, dermatitis exfoliative.
ⁿincludes pruritus, pruritus generalized.

Table 9 summarizes the laboratory abnormalities in REZUROCK.

TABLE 9
Selected Laboratory Abnormalities in Patients
with Chronic GVHD Treated with REZUROCK

| | REZUROCK | | |
|---|---|---|---|
| | 200 mg once daily | | |
| | Grade 0-1 | Grade 2-4 | Grade 3-4 |
| | Baseline | Max Post | Max Post |
| Parameter | (N) | (%) | (%) |
| Chemistry | | | |
| Phosphate Decreased | 76 | 28 | 7 |
| Gamma Glutamyl | 47 | 21 | 11 |
| Transferase Increased | | | |
| Calcium Decreased | 82 | 12 | 1 |
| Alkaline Phosphatase | 80 | 9 | 0 |
| Increased | | | |
| Potassium Increased | 82 | 7 | 1 |
| Alanine Aminotransferase | 83 | 7 | 2 |
| Increased | | | |
| Creatinine Increased | 83 | 4 | 0 |
| Hematology | | | |
| Lymphocytes Decreased | 62 | 29 | 13 |
| Hemoglobin Decreased | 79 | 11 | 1 |
| Platelets Decreased | 82 | 10 | 5 |
| Neutrophil Count | 83 | 8 | 4 |
| Decreased | | | |

7 DRUG INTERACTIONS

7.1 Effect of Other Drugs on REZUROCK

Strong CYP3A Inducers

Coadministration of REZUROCK with strong CYP3A inducers decreases belumosudil exposure [see Clinical Pharmacology (12.3)], which may reduce the efficacy of REZUROCK. Increase the dosage of REZUROCK when coadministered with strong CYP3A inducers [see Dosage and Administration (2.3)].

Proton Pump Inhibitors

Coadministration of REZUROCK with proton pump inhibitors decreases belumosudil exposure [see Clinical Pharmacology (12.3)], which may reduce the efficacy of REZUROCK. Increase the dosage of REZUROCK when coadministered with proton pump inhibitors [see Dosage and Administration (2.3)].

8 USE IN SPECIFIC POPULATIONS

8.1 Pregnancy

Risk Summary

Based on findings from animal studies and the mechanism of action [see Clinical Pharmacology (12.1)], REZUROCK can cause fetal harm when administered to pregnant women. There are no available human data on REZUROCK use in pregnant women to evaluate for a drug-associated risk. In animal reproduction studies, administration of belumosudil to pregnant rats and rabbits during the period of organogenesis resulted in adverse developmental outcomes, including alterations to growth, embryo-fetal mortality, and embryo-fetal malformations at maternal exposures (AUC) approximately ≥3-(rat) and ≥0.07 (rabbit) times the human exposure (AUC) at the recommended dose (see Animal Data). Advise pregnant women and females of reproductive potential of the potential risk to the fetus. In the U.S. general population, the estimated background risk of major birth defects and miscarriage in clinically recognized pregnancies is 2 to 4% and 15 to 20%, respectively.

Data

Animal Data

Embryo-fetal development studies were conducted in rats with administration of belumosudil to pregnant animals during the period of organogenesis at oral doses of 25, 50, 150, and 300 mg/kg/day in a pilot study and doses of 15, 50, and 150 mg/kg/day in a pivotal study. In the pilot study, maternal toxicity and embryo-fetal developmental effects were observed. Maternal toxicity (reduced body weight gain) occurred at 150 and 300 mg/kg/day doses. Increased post-implantation loss occurred at 50 and 300 mg/kg/day. Fetal-malformations were observed at ≥50 mg/kg/day and included absence of anus and tail, omphalocele, and dome shaped head. The exposure (AUC) at 50 mg/kg/day in rats is approximately 3 times the human exposure at the recommended dose of 200 mg.

In an embryo-fetal developmental study in rabbits, pregnant animals administered oral doses of belumosudil at 50, 125, and 225 mg/kg/day during the period of organogenesis resulted in maternal toxicity and embryo-fetal developmental effects. Maternal toxicity (body weight loss and mortality) was observed at doses ≥125 mg/kg/day. Embryo-fetal effects were observed at doses ≥50 mg/kg/day and included spontaneous abortion, increased post-implantation loss, decreased percentage of live fetuses, malformations, and decreased fetal body weight. Malformations included those in the tail (short), ribs (branched, fused or deformed), sternebrae (fused), and neural arches (fused, misaligned, and deformed). The exposure (AUC) at 50 mg/kg/day in rabbits is approximately 0.07 times the human exposure at the recommended dose of 200 mg.

8.2 Lactation

Risk Summary

There are no data available on the presence of belumosudil or its metabolites in human milk or the effects on the breastfed child, or milk production. Because of the potential for serious adverse reactions from belumosudil in the breastfed child, advise lactating women not to breastfeed during treatment with REZUROCK and for at least one week after the last dose.

8.3 Females and Males of Reproductive Potential

REZUROCK can cause fetal harm when administered to a pregnant woman [see Use in Specific Populations (8.1)].

### Pregnancy Testing

Verify the pregnancy status of females of reproductive potential prior to initiating treatment with REZUROCK. Contraception Females

Advise females of reproductive potential to use effective contraception during treatment with REZUROCK and for at least one week after the last dose of REZUROCK. If this drug is used during pregnancy or if the patient becomes pregnant while taking this drug, the patient should be informed of the potential hazard to a fetus.

### Males

Advise males with female partners of reproductive potential to use effective contraception during treatment with REZUROCK and for at least one week after the last dose of REZUROCK.

### Infertility

Females

Based on findings from rats, REZUROCK may impair female fertility. The effect on fertility is reversible [see Nonclinical Toxicology (13.1)].

### Males

Based on findings from rats and dogs, REZUROCK may impair male fertility. The effects on fertility are reversible [see Nonclinical Toxicology (13.1)].

8.4 Pediatric Use

The safety and effectiveness of REZUROCK have been established in pediatric patients 12 years and older. Use of REZUROCK in this age group is supported by evidence from adequate and well-controlled studies of REZUROCK in adults with additional population pharmacokinetic data demonstrating that age and body weight had no clinically meaningful effect on the pharmacokinetics of drug substance, that the exposure of drug substance is expected to be similar between adults and pediatric patients age 12 years and older, and that the course of disease is sufficiently similar in adult and pediatric patients to allow extrapolation of data in adults to pediatric patients.

The safety and effectiveness of REZUROCK in pediatric patients less than 12 years old have not been established.

8.5 Geriatric Use

Of the 186 patients with chronic GVHD in clinical studies of REZUROCK, 26% were 65 years and older. No clinically meaningful differences in safety or effectiveness of REZUROCK were observed in comparison to younger patients.

11 DESCRIPTION

Belumosudil is a kinase inhibitor. The active pharmaceutical ingredient is belumosudil mesylate with the molecular formula C27H28N605S and the molecular weight is 548.62 g/mol. The chemical name for belumosudil mesylate is 2-{3-[4-(1H-indazol-5-ylamino)-2-quinazolinyl] phenoxy}-N-(propan-2-yl) acetamide methanesulfonate (1:1). The chemical structure is as follows:

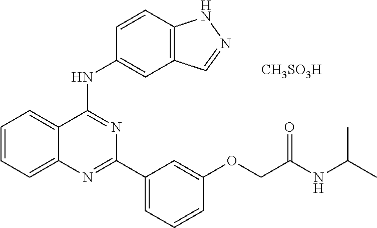

Belumosudil mesylate is a yellow powder that is practically insoluble in water, slightly soluble in methanol and DMF and soluble in DMSO.

REZUROCK tablets are for oral administration. Each tablet contains 200 mg of the free base equivalent to 242.5 mg of belumosudil mesylate. The tablet also contains the following inactive ingredients: microcrystalline cellulose, hypromellose, croscarmellose sodium, colloidal silicon dioxide, and magnesium stearate.

The tablet film consists of polyvinyl alcohol, polyethylene glycol, talc, titanium dioxide and yellow iron oxide.

12 CLINICAL PHARMACOLOGY

12.1 Mechanism of Action

Belumosudil is an inhibitor of rho-associated, coiled-coil containing protein kinase (ROCK) which inhibits ROCK2 and ROCK1 with IC50 values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of chronic GVHD.

12.2 Pharmacodynamics

Belumosudil exposure-response relationships and the time course of pharmacodynamic response are not established.

12.3 Pharmacokinetics

The following pharmacokinetic parameters are presented for chronic GVHD patients administered belumosudil 200 mg once daily, unless otherwise specified. The mean (% coefficient of variation, % CV) steady-state AUC and Cₘₐₓ of belumosudil was 22700 (48%) hong/mL and 2390 (44%) ng/mL, respectively. Belumosudil Cₘₐₓ and AUC increased in an approximately proportional manner over a dosage range of 200 and 400 mg (1 to 2 times once daily recommended dosage). The accumulation ratio of belumosudil was 1.4.

### Absorption

Median Tₘₐₓ of belumosudil at steady state was 1.26 to 2.53 hours following administration of 200 mg once daily or twice daily in patients. The mean (% CV) bioavailability was 64% (17%) following a single belumosudil dose in healthy subjects.

### Effect of Food

Belumosudil Cₘₐₓ and AUC increased 2.2 times and 2 times, respectively, following administration of a single belumosudil dose with a high-fat and high-calorie meal (800 to 1,000 calories with approximately 50% of total caloric content of the meal from fat) compared to the fasted state in healthy subjects. Median Tₘₐₓ was delayed 0.5 hours.

### Distribution

The geometric mean volume of distribution after a single dose of belumosudil in healthy subjects was 184 L (geo CV % 67.7%).

Belumosudil binding to human serum albumin and human 1-acid glycoprotein was 99.9% and 98.6%, respectively, in vitro.

### Elimination

The mean (% CV) elimination half-life of belumosudil was 19 hours (39%), and clearance was 9.83 L/hours (46%) in patients.

### Metabolism

Belumosudil is primarily metabolized by CYP3A4 and to a lesser extent by CYP2C8, CYP2D6, and UGT1A9, in vitro.

### Excretion

Following a single oral dose of radiolabeled belumosudil in healthy subjects, 85% of radioactivity was recovered in feces (30% as unchanged) and less than 5% in urine.

### Specific Populations

No clinically significant differences in belumosudil pharmacokinetics were observed with regard to age (18 to 77 years), sex, weight (38.6 to 143 kg), or mild to moderate renal impairment (eGFR ≥60 and <90 mL/min/1.72m2 to eGFR ≥30 and <60 mL/min/1.72m2). The effect of severe renal impairment on the pharmacokinetics of belumosudil has not been studied.

### Drug Interaction Studies

Clinical Studies and Model-Informed Approaches Effects of Other Drugs on Belumosudil

Strong Cytochrome P450 (CYP) 3A Inhibitors: There was no clinically meaningful effect on belumosudil exposure when coadministered with itraconazole in healthy subjects.

Strong CYP3A Inducers: Coadministration of rifampin decreased belumosudil Cₘₐₓ by 59% and AUC by 72% in healthy subjects.

Moderate CYP3A Inducers: Coadministration of efavirenz is predicted to decrease belumosudil Cₘₐₓ by 32% and AUC by 35% in healthy subjects.

Proton Pump Inhibitors: Coadministration of rabeprazole decreased belumosudil Cₘₐₓ by 87% and AUC by 80%, and omeprazole decreased belumosudil Cₘₐₓ by 68% and AUC by 47% in healthy subjects.

### Effects of Belumosudil on Other Drugs

CYP3A Substrates: Coadministration of belumosudil is predicted to increase midazolam (a sensitive CYP3A substrate) Cₘₐₓ and AUC approximately 1.3-and 1.5-fold, respectively.

CYP2C9 Substrates: Coadministration of belumosudil is not expected to have clinically meaningful effect on the exposure of CYP2C9 substrates (such as warfarin).

CYP2C8 Substrates: Coadministration of belumosudil is not expected to have clinically meaningful effect on the exposure of CYP2C8 substrates that are not an OATPIB1 substrate.

### In Vitro Studies

Transporter Systems: Belumosudil is a substrate of P-gp. Belumosudil inhibits BCRP, P-gp, and OATPIB1 at clinically relevant concentrations.

Enzymes Systems: Belumosudil is an inhibitor of CYP1A2, CYP2C19, CYP2D6, UGT1A1 and UGT1A9.

13 NONCLINICAL TOXICOLOGY

13.1 Carcinogenesis, Mutagenesis, Impairment of Fertility

Carcinogenicity studies have not been conducted with belumosudil.

Belumosudil was not genotoxic in an in vitro bacterial mutagenicity (Ames) assay, in vitro chromosome aberration assay in human peripheral blood lymphocytes (HPBL) or an in vivo rat bone marrow micronucleus assay.

In a combined male and female rat fertility study, belumosudil-treated male animals were mated with untreated females, or untreated males were mated with belumosudil-treated females. Belumosudil was administered orally at doses of 50, 150 or 275 mg/kg/day to male rats 70 days prior to and throughout the mating period, and to female rats 14 days prior to mating and up to Gestation Day 7. At the dose of 275 mg/kg/day, adverse findings in female rats (treated with belumosudil or untreated but mated with treated males) included increased pre-or post-implantation loss and decreased number of viable embryos. Administration of belumosudil to male rats at a dose of 275 mg/kg/day resulted in abnormal sperm findings (reduced motility, reduced count, and increased percentage of abnormal sperm), and testes/epididymis organ changes (reduced weight and degeneration).

Fertility was reduced in both treated males or females at the 275 mg/kg/day dose and reached statistical significance in males. Adverse changes in male and female reproductive organs also occurred in general toxicology studies; findings included spermatozoa degeneration at a belumosudil dose of 35 mg/kg/day in dogs and decreased follicular development in ovaries at 275 mg/kg/day in rats. Changes were partially or fully reversed during the recovery period. The exposure (AUC) at the doses of 35 mg/kg/day in dogs, and 275 mg/kg/day in rats is 0.5 times and 8-9 times, respectively, the clinical exposure at the recommended dose of 200 mg daily.

14 CLINICAL STUDIES

14.1 Chronic Graft versus Host Disease

Study KD025-213 (NCT03640481) was a randomized, open-label, multicenter study of REZUROCK for treatment of patients with chronic GVHD who had received 2 to 5 prior lines of systemic therapy and required additional treatment. Patients were excluded from the studies if platelets were <50×109/L; absolute neutrophil count <1.5×109/L; AST or ALT >3 ×ULN; total bilirubin >1.5 ×ULN; QTc (F)>480 ms; eGFR <30 mL/min/1.73 m2; or FEV1 ≤39%. There were 66 patients treated with REZUROCK 200 mg taken orally once daily. Concomitant treatment with supportive care therapies for chronic GVHD was permitted. Concomitant treatment with GVHD prophylaxis and standard care systemic chronic GVHD therapies was permitted as long as the subject has been on a stable dose for at least 2 weeks prior to study. Initiation of new systemic chronic GVHD therapy while on study was not permitted.

Demographics and baseline characteristics are summarized in Table 10.

TABLE 10
Demographics and Baseline Characteristics
of Patients with Chronic GVHD

| | REZUROCK |
|---|---|
| | 200 mg once |
| | daily (N = 65) |
| Age, Median, Years (minimum, maximum) | 53 (21, 77) |
| Age ≥65 Years, n (%) | 17 (26) |
| Male, n (%) | 42 (65) |
| Race, n (%) | |
| White | 54 (83) |
| Black | 6 (9) |
| Other or Not Reported | 5 (8) |
| Median (range) time (months) from Chronic | 25.3 (1.9, 162.4) |
| GVHD Diagnosis | |
| ≥4 Organs Involved, n (%) | 31 (48) |
| Median (range) Number of Prior Lines of Therapy | 3 (2, 6) |
| Number of Prior Lines of Therapy, n (%) | |
| 2 | 23 (35) |
| 3 | 12 (19) |
| 4 | 15 (23) |
| ≥5 | 15 (23) |
| Prior chronic GVHD treatment with ibrutinib, | 21 (32) |
| n (%) | |
| Prior chronic GVHD treatment with ruxolitinib, | 20 (31) |
| n (%) | |
| Refractory to Last Therapy, n (%^{a}) | 43/55 (78) |
| Severe chronic GVHD, n (%) | 46 (71) |
| Median (range) Global Severity Rating | 7 (2, 9) |
| Median (range) Lee Symptom Scale Score at | 27 (7, 56) |
| baseline | |
| Median (range) Corticosteroid dose at baseline | 0.19 (0.03, 0.95) |
| (PE/kg)^{b} | |

^{a}Denominator excludes patients with unknown status
^{b}Prednisone equivalents/kilogram

The efficacy of REZUROCK was based on overall response rate (ORR) through Cycle 7 Day 1 where overall response included complete response or partial response according to the 2014 NIH Response Criteria. The ORR results are presented in Table 11. The ORR was 75% (95% CI: 63, 85). The median duration of response, calculated from first response to progression, death, or new systemic therapies for chronic GVHD, was 1.9 months (95% CI: 1.2, 2.9). The median time to first response was 1.8 months (95% CI: 1.0, 1.9). In patients who achieved response, no death or new systemic therapy initiation occurred in 62% (95% CI: 46, 74) of patients for at least 12 months since response.

TABLE 11
Overall Response Rate through Cycle 7 Day 1 for
Patients with Chronic GVHD in Study KD025-213

| | REZUROCK 200 mg |
|---|---|
| | once daily (N = 65) |
| Overall Response Rate (ORR) | 49 (75%) |
| 95% Confidence Interval^{a} | (63%, 85%) |
| Complete Response | 4 (6%) |
| Partial Response | 45 (69%) |

^{a}Estimated using Clopper-Pearson method

ORR results were supported by exploratory analyses of patient-reported symptom bother which showed at least a 7-point decrease in the Lee Symptom Scale summary score through Cycle 7 Day 1 in 52% (95% CI: 40, 65) of patients.

16 HOW SUPPLIED/STORAGE AND HANDLING

REZUROCK 200 mg tablets are supplied as pale yellow film-coated oblong tablets containing 200 mg of belumosudil (equivalent to 242.5 mg belumosudil mesylate). Each tablet is debossed with “KDM” on one side and “200” on the other side and is packaged as follows: 200 mg tablets in 30 count bottle: NDC 79802-200-30

Store at room temperature, 20° C. to 25° C. (68° F. to 77° F.); excursions permitted from 15° C. and 30° C. (59° F. to 86° F.) [see USP Controlled Room Temperature].

Dispense to patient in original container only. Store in original container to protect from moisture. Replace cap securely each time after opening. Do not discard desiccant.

17 PATIENT COUNSELING INFORMATION

Advise the patient to read the FDA-approved patient labeling (Patient Information).

Embryo-fetal Toxicity:
Advise pregnant women and females of reproductive potential of the potential risk to a fetus. Advise females of reproductive potential to inform their healthcare provider of a known or suspected pregnancy [see Warnings and Precautions (5.1), Use in Specific Populations (8.1, 8.3)].
Advise females of reproductive potential to use effective contraceptive during treatment with REZUROCK and for at least one week after the last dose [see Warnings and Precautions (5.1)].
Advise males with female partners of reproductive potential to use effective contraceptive during treatment with REZUROCK and for at least one week after the last dose [see Use in Specific Populations (8.3)].

### Lactation

Advise women not to breastfeed during treatment with REZUROCK and for at least one week after the last dose [see Use in Specific Populations (8.2)].

### Infertility

Advise males and females of reproductive potential that REZUROCK may impair fertility [see Use in Specific Populations (8.3)].

### Administration

Inform patients to take REZUROCK orally once daily with food according to their physician's instructions and that the oral dosage (tablets) should be swallowed whole with a glass of water without cutting, crushing or chewing the tablets approximately the same time each day [see Dosage and Administration (2.1)].

Advise patients that in the event of a missed daily dose of REZUROCK, it should be taken as soon as possible on the same day with a return to the normal schedule the following day. Patients should not take extra doses to make up the missed dose [see Dosage and Administration (2.1)].

### Drug Interactions

Advise patients to inform their health care providers of all concomitant medications, including prescription medicines, over-the-counter drugs, vitamins, and herbal products [see Drug Interactions (Z)].

TABLE 12
Patient Information Patient INFORMATION REZUROCK
(REZ-ur-ok) (belumosudil) tablets

What is REZUROCK?
REZUROCK is a prescription medicine used to treat adults and children 12 years of age and
older with chronic graft- versus-host disease (chronic GVHD) after you have received at least
2 prior treatments (systemic therapy) and they did not work.
It is not known if REZUROCK is safe and effective in children less than 12 years old.
Before taking REZUROCK, tell your healthcare provider about all of your medical
conditions, including if you:
have kidney or liver problems.
are pregnant or plan to become pregnant. REZUROCK can harm your unborn baby. If you
are able to become pregnant, your healthcare provider will do a pregnancy test before
starting treatment with REZUROCK. Tell your healthcare provider if you become pregnant
or think you may be pregnant during treatment with REZUROCK.
Females who can become pregnant should use effective birth control during treatment
with REZUROCK and for at least 1 week after the last dose.
Males with female partners who can become pregnant should use effective birth
control during treatment with REZUROCK and for at least 1 week after the last dose.
are breastfeeding or plan to breastfeed. It is not known if REZUROCK passes into breast
milk. Do not breastfeed during treatment with REZUROCK and for at least 1 week after
the last dose.
Tell your healthcare provider about all the medicines you take, including prescription and
over-the-counter medicines, vitamins, and herbal supplements. REZUROCK may affect the
way other medicines work, and other medicines may affect the way REZUROCK works.
Know the medicines you take. Keep a list of them to show your healthcare provider and
pharmacist when you get a new medicine.
How should I take REZUROCK?
Take REZUROCK exactly as your healthcare provider tells you to take it.
Do not change your dose or stop taking REZUROCK without first talking to your
healthcare provider.
Take REZUROCK 1 time a day with a meal.
Take REZUROCK at about the same time each day.
Swallow REZUROCK tablets whole with a glass of water.
Do not cut, crush, or chew REZUROCK tablets.
Your healthcare provider will do blood tests to check your liver at least 1 time a month
during treatment with REZUROCK.
If you miss a dose of REZUROCK, take it as soon as you remember on the same day.
Take your next dose of REZUROCK at your regular time on the next day. Do not take
extra doses of REZUROCK to make up for a missed dose.
If you take too much REZUROCK, call your healthcare provider or go to the nearest
hospital emergency room right away.
What are the possible side effects of REZUROCK?
The most common side effects of REZUROCK include:
infections □ swelling
tiredness or weakness □ bleeding
nausea □ stomach (abdominal) pain
diarrhea □ muscle or bone pain
shortness of breath □ headache
cough □ high blood pressure
Your healthcare provider may change your dose of REZUROCK, temporarily stop, or
permanently stop treatment with REZUROCK if you have certain side effects.
REZUROCK may affect fertility in males and females. Talk to your healthcare provider if this
is a concern for you. These are not all the possible side effects of REZUROCK.
Call your doctor for medical advice about side effects. You may report side effects to FDA at
1-800-FDA-1088. You may also report side effects to Kadmon Pharmaceuticals, LLC at 1-
877-377-7862.
How should I store REZUROCK?
Store REZUROCK at room temperature between 68° F. to 77° F. (20° C. to 25° C.).
Keep REZUROCK in its original container. The REZUROCK bottle contains a desiccant
packet to help keep your tablets dry (protect from moisture). Keep the desiccant in the
bottle.
Tightly close the REZUROCK bottle after you take your dose.
Keep REZUROCK and all medicines out of the reach of children.
General information about the safe and effective use of REZUROCK.
Medicines are sometimes prescribed for purposes other than those listed in a Patient
Information leaflet. Do not use REZUROCK for a condition for which it was not prescribed.
Do not give REZUROCK to other people, even if they have
the same symptoms that you have. It may harm them. You can ask your pharmacist or
healthcare provider for information about REZUROCK that is written for health professionals.
What are the ingredients in REZUROCK? Active ingredient: belumosudil mesylate
Inactive ingredients:
Tablet core: microcrystalline cellulose, hypromellose, croscarmellose sodium, colloidal silicon
dioxide, and magnesium stearate.
Tablet coating: polyvinyl alcohol, polyethylene glycol, talc, titanium dioxide and yellow iron
oxide.

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

1-39. (canceled)

40. A method of treating chronic graft-versus-host disease (cGVHD) in a subject in need thereof, comprising administering 2-{3-[4-(1H-indazol-5-ylamino)-2-quinazolinyl] phenoxy}-N-(propan-2-yl) acetamide or a pharmaceutically acceptable salt thereof (Compound), wherein the subject ingests a meal within about one hour or less prior to ingesting the Compound.

41. The method of claim 40, wherein the Compound comprises a mesylate salt.

42. The method of claim 40, wherein the subject has failed at least two prior lines of systemic therapy for the cGVHD.

43. The method of claim 42, wherein the prior lines of systemic therapy are selected from prednisone, tacrolimus, ECP, sirolimus, ibruitinib, ruxolitinib, MMF, rituximab, MTX, cyclosporine, imatinib, ixazomib, and ofatumumab.

44. The method of claim 40, wherein the daily dose of the Compound is equivalent to an amount of the free base of the Compound that is in the range of about 200 mg to about 800 mg.

45. The method of claim 44, wherein the daily dose of the Compound is equivalent to an amount of the free base of the Compound that is about 200 mg.

46. The method of claim 44, wherein the daily dose of the Compound is equivalent to an amount of the free base of the Compound that is about 400 mg.

47. The method of claim 40, wherein the subject ingests a meal within about thirty minutes of ingesting the Compound.

48. The method of claim 40, wherein the meal is a high-fat meal.

49. A method of treating chronic graft-versus-host disease (cGVHD) in a subject in need thereof, comprising the steps of:
(a) identifying the subject as having ingested a meal; and
(b) administering 2-{3-[4-(1H-indazol-5-ylamino)-2-quinazolinyl] phenoxy}-N-(propan-2-yl) acetamide or a pharmaceutically acceptable salt thereof (Compound) to the subject, wherein the meal was ingested about one hour or less prior to administering the Compound.

50. The method of claim 49, wherein the Compound comprises a mesylate salt.

51. The method of claim 49, wherein the subject has failed at least two prior lines of systemic therapy for the cGVHD.

52. The method of claim 51, wherein the prior lines of systemic therapy are selected from prednisone, tacrolimus, ECP, sirolimus, ibruitinib, ruxolitinib, MMF, rituximab, MTX, cyclosporine, imatinib, ixazomib, and ofatumumab.

53. The method of claim 49, wherein the daily dose of the Compound is equivalent to an amount of the free base of the Compound that is in the range of about 200 mg to about 800 mg.

54. The method of claim 53, wherein the daily dose of the Compound is equivalent to an amount of the free base of the Compound that is about 200 mg.

55. The method of claim 53, wherein the daily dose of the Compound is equivalent to an amount of the free base of the Compound that is about 400 mg.

56. The method of claim 49, wherein the meal was ingested about thirty minutes prior to administering the Compound.

57. The method of claim 49, wherein the meal is a high-fat meal.

58. A method of treating chronic graft-versus-host-disease (cGVHD) in a subject in need thereof, who is concurrently taking a CYP3A Inducer or Proton Pump Inhibitor (PPI), comprising administering to the subject a protocol to mediate the food effect in combination with the exposure reduction effects of the CYP3A Inducer or PPI.
